(19) United States
(12) Patent Application Publication
(10) Pub. No.: US 2025/0228771 A1
(43) Pub. Date: Jul. 17, 2025

(54) ALKALINE INTRAORAL PRODUCTS

(71) Applicant: Emplicure AB (publ), Uppsala (SE)

(72) Inventors: Håkan ENGQVIST, Uppsala (SE); Jesper LÖÖF, Bälinge (SE)

(73) Assignee: Emplicure AB (publ), Uppsala (SE)

(21) Appl. No.: 18/852,986

(22) PCT Filed: Apr. 3, 2023
(86) PCT No.: PCT/EP2023/058720

§ 371 Date: Sep. 30, 2024

(30) Foreign Application Priority Data
Apr. 1, 2022 (GB) 2204804.5
Jun. 15, 2022 (GB) 2208814.0

(51) Int. Cl. A61K 9/00; A61K 31/216; A61K 31/4468; A61K 31/465; A61K 31/485; A61K 47/02

(52) U.S. Cl. CPC A61K 9/006; A61K 31/216; A61K 31/4468; A61K 31/465; A61K 31/485; A61K 47/02

(57) ABSTRACT

There is provided an intraoral composition comprising an alkaline active pharmaceutical agent, or a salt thereof, and a chemically bonded ceramic system formed from a substance selected from the group consisting of calcium aluminates, calcium silicates, calcium phosphates and mixtures thereof. The intraoral composition may be held in the mouth where it releases the alkaline active pharmaceutical agent and contributes to providing an environment that facilitates uptake into the blood stream. Active pharmaceutical agents of particular interest include nicotine and opioid analgesics.

### FIELD OF THE INVENTION

The invention relates to new formulations for intraoral delivery of active substances.

The invention also relates to a method of manufacturing products suitable for intraoral delivery. The formulations may contain nicotine or another alkaline active substance and be employed in recreational use products as well as in medicine, e.g. aiding smoking cessation, treating nicotine dependence or alleviating the symptoms of nicotine dependence, or treating pain.

### BACKGROUND

Intraoral administration of active substances can be challenging for a number of reasons. In the case of administration of nicotine, an alkaloid, which is often presented in buccal pouches, the pH in the liquid in which the nicotine is dissolved needs to be in the range of 8-8.5, and possibly higher, in order to obtain sufficient bioavailability. This ensures that the nicotine is predominantly present in a unionized state as a molecule in this state will pass through the oral mucosa more readily than a charged nicotine ion. The choice of pH range reflects the upper pKa value for nicotine of about 8.02 (Tomar S. L. et al., Tobacco Control 1997; 6:219-225). An upper pH limit is set by what is acceptable from a user perspective in terms of sensation when the pouches are used.

Various nicotine replacement therapy products are commercially available, including patches, gums, lozenges, nicotine pouches, sublingual tablets, inhalers and nasal sprays (U. Wadgave et al., Int J Health Sci (Qassim), 2016 July; 10(3): 425-435). Nicotine replacement therapies such as these are useful in helping tobacco users to overcome difficulties arising from nicotine withdrawal as they attempt to reduce their exposure to tobacco products, particularly cigarettes.

Some nicotine delivery systems are also used by individuals to provide pleasurable sensations resulting from nicotine uptake. Intraoral products that are used in this way include nicotine pouches and snus, the latter being particularly popular in Norway and Sweden. Snus is a tobacco-containing product that is typically provided as either loose snus or portion snus. Snus products are also a convenient and safer alternative to cigarettes as they offer a source of nicotine which does not expose the user to some of risks associated with cigarette smoking, such as harmful substances found in cigarette smoke (e.g. carbon monoxide, benzene and formaldehyde) and in the cigarette itself (e.g. vinyl chloride that is used in cigarette filters).

Loose snus is a moist powder which can be portioned and packed into a cylindrical or spherical shape with the fingertips or a purpose-made device. The shaped mass of snus is then placed under their upper lip. Over time, the demand for loose snus has been replaced by portioned varieties, which benefits users due to easy handling and their more discreet nature.

Portion snus is packaged as a moist powder in small teabag-like sachets, which are also intended for positioning under the upper lip. Like snus, portion snus is typically held in the mouth for a period lasting between five minutes and one hour and nicotine is released throughout this time.

Portion snus is available in three different sizes: mini, normal/large (most common) and maxi. Mini portions typically weigh close to 0.5 g, normal (large) portions weigh around 0.8 to 1 gram, and maxi portions weigh up to 1.7 g. Some manufacturers also offer the choice of “regular” and “long” versions of the normal size sachet, which are similar in content weight. These long portions differ from traditional sachets in that they are slimmer but longer, in order to fit against the gums more comfortably. Tobacco-containing snus products often require refrigerated storage conditions, primarily to slow the drying of the tobacco. Refrigeration is also commonly used for tobacco-free nicotine pouches products in order to improve the stability of the product.

Nicotine pouches are white pre-portioned pouches containing either tobacco-derived nicotine or synthetic nicotine, potentially in salt form, but no tobacco leaf, ground tobacco (i.e. dust), or tobacco stem. They are described as either similar to snus, or a tobacco-free version of snus. Non-tobacco-based nicotine pouches are commercially available, and one of the first examples of this was ZYN®, a product marked by Swedish Match® (N. Plurphanswat et al., The American Journal on Addictions, 29: 279-286, 2020; Lunell E., et al., Nicotine & Tobacco Research, 2020, 1757-1763). A number of cellulose-containing commercial products are available that deliver nicotine. However, when placed in the mouth, the cellulose can interact with water to form a slimy lump in the consumer's mouth, which negatively affects the user's flavour experience and will therefore likely affect the release of nicotine.

Nicotine pouches have been approved by e.g. the Norwegian Medicines Agency for smoking cessation and are sold as a nicotine replacement therapy. Nicotine pouches are also increasingly used recreationally as an alternative to tobacco products such as cigarettes and snus. Nicotine pouches may be dry or moist. Pouches that are moist are generally feel more comfortable in the mouth, and often provide for quicker release of nicotine. Moist pouches typically contain glycerol.

To use a nicotine pouch, the user puts a pouch in contact with the inside surface of the mouth, typically between the upper lip and gum, behind the lower lip or against the cheek, and leaves it there while the nicotine and taste is being released, much like portion snus. The pouch is typically kept in the mouth for a period of from five minutes to one hour and are then disposed of when finished.

Fast-releasing oral nicotine products are disclosed in the international patent application with the publication no. WO 2019/110073. Pouches containing nicotine in free salt form are disclosed in U.S. Pat. No. 9,161,908.

The delivery of nicotine through the oral mucosa (particularly sublingual and buccal mucosa) can be greatly enhanced by increasing the pH of the oral environment above the normal level, typically to around pH 8-9 (See Ciolino L A, McCauley H A, Fraser D B, Wolnik K A. J. Anal. Toxicol. 2001; 25:15-25, and Tomar S. L. et al. Tobacco Control 1997; 6:219-225). A pH regulating agent or suitable buffering agent may be incorporated into an intraoral product in order to achieve this pH increase. In most nicotine pouches, the desired pH is achieved by adding sodium carbonate or sodium bicarbonate, both of which dissolve when the pouch is used and thus create the alkaline pH. However, the use of these substances as sources of alkalinity presents challenges, for example concerning whether this alkaline system generates enough alkalinity and whether it has any buffer capacity to make sure that the pH at the site of absorption is kept at the appropriate levels for a sufficient period of time. Ideally, the alkaline environment needs to be maintained whilst the pouch is in use, and at least as long as there is nicotine is being released from the pouch.

Commercially available nicotine pouches typically have a release profile in which a significant fraction of the nicotine is released in a short time and may lead to nicotine being swallowed and/or cause saturation of the mucosa. It has been speculated that this leads to a significant amount of the available nicotine being washed away by saliva and swallowed if the mucosa and immediately surrounding saliva is saturated. However, controlling the total amount and rate of release of nicotine from an intraoral product to provide long lasting therapeutic and/or non-therapeutic effects presents further challenges, for example in sustaining an appropriate alkaline environment in the mouth.

Pharmaceuticals are also often formulated as gums, lozenges, pouches, sublingual tablets, and other forms that are intended to provide release of the active substance in the mouth. Examples of commonly prescribed sublingual tablets include nitroglycerin, loratadine, mirtazapine, and rizatriptan, though many drugs can be absorbed through sublingual administration, including opioid analgesics, cardiovascular drugs, barbiturates, benzodiazepines, THC, CBD and increasingly, vitamins.

As with nicotine, uptake of the active substance through the oral mucosa can often be improved if an appropriate pH environment at the site of absorption in the mouth can be obtained and sustained.

There is an unmet need for oral transmucosal (or intraoral) products offering controlled release of nicotine and other pharmaceutical substances and which also provide and maintain a suitably alkaline environment to ensure adequate uptake of those substances into the bloodstream.

We have now devised a novel formulation for substances, like nicotine, for which uptake is improved when placed in a mildly alkaline environment. These intraorally deliverable products have advantageous properties and solve one or more of the problems associated with existing formulations intended for intraoral delivery.

The listing or discussion of an apparently prior-published document in this specification should not necessarily be taken as an acknowledgement that the document is part of the state of the art or is common general knowledge.

### DISCLOSURE OF THE INVENTION

According to a first aspect of the invention, there is provided an intraoral composition comprising an alkaline active pharmaceutical agent, or a salt thereof, and a chemically bonded ceramic system formed from a substance selected from the group consisting of calcium aluminates, calcium silicates, calcium phosphates and mixtures thereof.

These intraoral compositions are hereinafter referred to as “compositions of the invention”.

Chemically bonded ceramic systems formed from a substance selected from the group consisting of calcium aluminates, calcium silicates, calcium phosphates and mixtures thereof are hereinafter referred to as alkaline bioceramics”.

We have advantageously found that alkaline bioceramics can provide a stable and sustained source of alkalinity. This is particularly useful (e.g. in medicine) for the administration of chemical agents for which uptake into the bloodstream is enhanced under alkaline conditions. Examples of such chemical agents include nicotine and opioid analgesics. The alkaline bioceramics may also advantageously be used as a carrier for the alkaline active pharmaceutical agent in the composition, though this is not essential in compositions of the invention. When used as a carrier, alkaline bioceramics can be used to achieve a high degree of loading of the alkaline active pharmaceutical agent into the pores of the chemically bonded ceramic system whilst still enabling the product to deliver the agent through the oral mucosa at a satisfactory rate and to a satisfactory extent once in the mouth.

Without wishing to be bound by theory, it is believed that alkaline bioceramics are a source of alkalinity because they act as a supply of hydroxide ions. These hydroxide ions can leach out of the chemically bonded ceramic system when it is placed in an aqueous environment, thereby altering the pH of that aqueous system. This leaching can occur gradually over an extended period of time due, in part, to the fact that these alkaline bioceramics are typically porous systems such that water is able to penetrate the increasingly deeply into the pores over time to thereby draw out more hydroxide ions.

The intraoral compositions of the invention are designed to be held in the mouth of a person where they come into contact with saliva. The pH of normal human saliva is around 6.8. When placed in the mouth, the compositions of the invention are capable of altering the pH locally at the site of administration, typically by increasing the pH of that environment above normal. The magnitude of that change may be altered by adjusting the intraoral composition. For example, the amount of alkaline bioceramic may be increased or decreased. Alternatively, the porosity of that material may also be varied to allow for release of larger or smaller quantities of hydroxide ions. In one embodiment, the composition of the invention is capable of providing a pH of at least 8 upon contact with saliva. It is preferred that the pH of the saliva inside and close to the composition in use_is not increased too far to avoid damage to the tissues in the mouth. In a further embodiment therefore, the composition of the invention is capable of providing a pH of up to 9.5 (e.g. up to 9, up to 8.8 or up to 8.5), upon contact with saliva.

As a result of their ability to increase salivary pH locally at the site of administration, the compositions of the invention are useful in the delivery of chemical agents for which uptake into the bloodstream is enhanced under alkaline conditions. For example, Tomar S. L. et al. (Tobacco Control 1997; 6:219-225) reported that increased alkalinity promotes the absorption of nicotine via the oral mucosa and increases its physiological effects. The term “alkaline active pharmaceutical agents” refers to basic active pharmaceutical agents that tend to become protonated under neutral or acidic conditions. An effective measure of alkalinity of any given active pharmaceutical agent is its acidity constant “Kₐ”, typically reported as the pKₐ. Alkaline active pharmaceutical agents that are particularly suited for use in the compositions of the invention include those which have a pKₐ of at least 7. Preferably, the pKₐ is in the range of 6 to 12, e.g. 7 to 10.

### Nicotine Products

One example of an alkaline active pharmaceutical agents that is particularly suited for use in the compositions of the invention is nicotine or a salt thereof. The pKₐ values of nicotine are about 3.2 and 8.02 (Tomar S. L. et al., Tobacco Control 1997; 6:219-225). Absorption of nicotine from the oral cavity, i.e. transmucosal uptake, to the systemic circulation is dependent on the local pH of the saliva inside and close to the composition in use. Nicotine will predominantly be absorbed through the mucosa when in the un-ionised form. That is, when the local pH is sufficiently alkaline to ensure that the nicotine is predominantly in the non-protonated form. Therefore, provision of a local pH which results in a high fraction of the nicotine being non-protonated enhances transport of nicotine across the oral mucosa and into the bloodstream. An intraoral composition that is capable of providing a pH in the range of 8 to 8.5 upon contact with saliva is particularly suited for use with nicotine (as well as other drugs with a similar degree of alkalinity). Thus in one embodiment, the intraoral composition is capable of providing a pH in the range of 8 to 8.5 upon contact with saliva. In another embodiment, the alkaline active pharmaceutical agents is not nicotine (or a salt thereof).

In one embodiment, the composition of the invention is prepared using a salt of nicotine, such as a nicotine bitartrate salt (e.g. nicotine bitartrate dihydrate). Other salt forms that may be mentioned include nicotine ascorbate, nicotine aspartate, nicotine benzoate, nicotine monotartrate, nicotine chloride (e.g., nicotine hydrochloride and nicotine dihydrochloride), nicotine citrate, nicotine fumarate, nicotine gentisate, nicotine lactate, nicotine mucate, nicotine laurate, nicotine levulinate, nicotine malate, nicotine perchlorate, nicotine pyruvate, nicotine salicylate, nicotine sorbate, nicotine succinate, nicotine zinc chloride, nicotine sulphate, nicotine tosylate and hydrates thereof (e.g., nicotine zinc chloride monohydrate). For the avoidance of doubt, where a formulation of the invention is prepared using a salt of nicotine, the nicotine may be present in the final formulation as nicotine free base, a salt of nicotine (e.g. nicotine bitartrate) or a mixture thereof. Furthermore, the nicotine in the formulation may be largely (e.g. at least 70%) amorphous.

Preferably, some or all of the nicotine is present in the compositions of the invention as a water-soluble form of nicotine. In this context, the term “water-soluble form” is understood as referring to a form of nicotine having a solubility in water of at least 10 g of salt per 100 mL water under ambient conditions, including temperature of 25° C., atmospheric pressure, and pH of 7. The nicotine present in the compositions of the invention is believed to exist as an (at least largely) amorphous material. At the start of the manufacturing process, the nicotine or salt thereof is preferably provided in the form of a crystalline solid because crystalline solids of high purity can be readily sourced.

Where the nicotine is to be incorporated into the pores of the alkaline bioceramic (as is discussed in more detail below), the process by which that chemically bonded ceramic systems is formed involves hydration of suitable precursor materials in the presence of nicotine (or a salt thereof) followed by a hardening step. During the process, the nicotine (or salt) is dissolved and/or diluted in the hydration liquid, and the subsequent drying step results in the precipitation of an amorphous nicotine product within pores in the chemically bonded ceramic system. This amorphous nicotine product may be a salt of nicotine. Powder X-ray crystallography can be used to detect the presence of crystalline components (such as nicotine salts), and other methods for detection and quantification of crystallinity and/or amorphicity are known in the art.

Compositions of the invention containing nicotine are intended to be used for both therapeutic and recreational purposes. The final formulations containing the compositions of the invention are intended for intraoral administration, and are referred to herein interchangeably as “intraoral products” and “oral products”. These offer an alternative to tobacco products (such as cigarettes and tobacco-containing snus) and can so be used to reduce a user's exposure to many of the potentially toxic components found in tobacco products. Nicotine is typically obtained from tobacco products, e.g. tobacco oil and other extracts. For example, the nicotine can be provided as a free base (e.g., as a mixture of nicotine free base and a porous particulate carrier such as microcrystalline cellulose), a nicotine salt (e.g., as nicotine tartrate or nicotine bitartrate or another organic acid salt of nicotine), as a resin complex of nicotine (e.g., nicotine polacrilex), or as a solvate or other suitable form.

### Other Active Pharmaceutical Agents

Other alkaline active pharmaceutical agents may also be used in the compositions of the invention as it is believed that they may similarly exhibit enhanced transport across the oral mucosa and into the bloodstream as a result of the local pH changes caused by the alkaline bioceramic in the composition. Intraoral compositions capable of providing pH values different from those described hereinabove in respect of nicotine may be required depending on the active pharmaceutical agent in question, and the skilled person would be able to select a suitable pH range accordingly. Typically, alkaline active pharmaceutical agents are low molecular weight organic compounds. These substances have a molecular weight that is small enough to allow for the possibility of diffusion across cell membranes (e.g. less than 1000 daltons), and also are sufficiently water soluble to enable them to dissolve in saliva. These agents also have at least one functional group which is alkaline in nature. Typically this is a nitrogen-containing group, such as a primary, secondary, tertiary or aromatic amine.

Other alkaline active pharmaceutical agents that may be employed in compositions of the invention preferably include active ingredients from various pharmacological classes, e.g. antibacterial agents, antihistamines and decongestants, anti-inflammatory agents, antiparasitics, antivirals, local anaesthetics, antifungals, amoebicidals or trichomonocidal agents, analgesics, antianxiety agents, anticlotting agents, antiarthritics, antiasthmatics, anticoagulants, anticonvulsants, antidepressants, antidiabetics, antiglaucoma agents, antimalarials, antimicrobials, antineoplastics, antiobesity agents, antipsychotics, antihypertensives, auto-immune disorder agents, anti-impotence agents, anti-Parkinsonism agents, anti-Alzheimer's agents, antipyretics, anticholinergics, anti-ulcer agents, blood-glucose-lowering agents, bronchodilators, central nervous system agents, cardiovascular agents, cognitive enhancers, contraceptives, cholesterol-reducing agents, agents that act against dyslipidermia, cytostatics, diuretics, germicidials, H2 blockers, proton pump inhibitors, hormonal agents, anti-hormonical agents, hypnotic agents, inotropics, muscle relaxants, muscle contractants, physic energizers, sedatives, sympathomimetics, vasodilators, vasocontrictors, tranquilizers, electrolyte supplements, vitamins, uricosurics, cardiac glycosides, membrane efflux inhibitors, membrane transport protein inhibitors, expectorants, purgatives, contrast materials, radiopharmaceuticals, imaging agents, peptides, enzymes, growth factors, vaccines, mineral trace elements.

However, preferred active pharmaceutical agents that may be employed in compositions of the invention include opioid analgesics. The term “opioid analgesic” will be understood by the skilled person to include any substance, whether naturally-occurring or synthetic, with opioid or morphine-like properties and/or which binds to opioid receptors, particularly the μ-opioid receptor, having at least partial agonist activity, thereby capable of producing an analgesic effect. The problems of potential formulation tampering and drug extraction by drug addicts are particularly prominent with opioids.

Opioid analgesics that may be mentioned include opium derivatives and the opiates, including the naturally-occurring phenanthrenes in opium (such as morphine, codeine, thebaine and Diels-Alder adducts thereof) and semisynthetic derivatives of the opium compounds (such as diamorphine, hydromorphone, oxymorphone, hydrocodone, oxycodone, etorphine, nicomorphine, hydrocodeine, dihydrocodeine, metopon, normorphine and N-(2-phenylethyl)normorphine). Other opioid analgesics that may be mentioned include fully synthetic compounds with opioid or morphine-like properties, including morphinan derivatives (such as racemorphan, levorphanol, dextromethorphan, levallorphan, cyclorphan, butorphanol and nalbufine); benzomorphan derivatives (such as cyclazocine, pentazocine and phenazocine); phenylpiperidines (such as pethidine (meperidine), fentanyl, alfentanil, sufentanil, remifentanil, ketobemidone, carfentanyl, anileridine, piminodine, ethoheptazine, alphaprodine, betaprodine, 1-methyl-4-phenyl-1,2,3,6-tetrahydropyridine (MPTP), diphenoxylate and loperamide), phenylheptamines or “open chain” compounds (such as methadone, isomethadone, propoxyphene and levomethadyl acetate hydrochloride (LAAM)); diphenylpropylamine derivatives (such as dextromoramide, piritramide, bezitramide and dextropropoxyphene); mixed agonists/antagonists (such as buprenorphine, nalorphine and oxilorphan) and other opioids (such as tilidine, tramadol and dezocine). Further opioid analgesics that may be mentioned include allylprodine, benzylmorphine, clonitazene, desomorphine, diampromide, dihydromorphine, dimenoxadol, dimepheptanol, dimethylthiambutene, dioxaphetyl butyrate, dipipanone, eptazocine, ethylmethylthiambutene, ethylmorphine, etonitazene, hydroxypethidine, levophenacylmorphan, lofentanil, meptazinol, metazocine, myrophine, narceine, norpipanone, papaveretum, phenadoxone, phenomorphan, phenoperidine and propiram.

More preferred opioid analgesics include buprenorphine, alfentanil, sufentanil, remifentanil and, particularly, fentanyl.

Active ingredients listed above may be formulated in compositions of the invention in any specific combination.

Active ingredients (including nicotine) may further be employed in salt form or any other suitable form, such as e.g. a complex, solvate or prodrug thereof, or in any physical form such as, e.g., in an amorphous state, as crystalline or part-crystalline material, as co-crystals, or in a polymorphous form or, if relevant, in any stereoisomeric form including any enantiomeric, diastereomeric or racemic form, or a combination of any of the above.

Pharmaceutically-acceptable salts of active ingredients that may be mentioned include acid addition salts and base addition salts. Such salts may be formed by conventional means, for example by reaction of a free acid or a free base form of an active ingredient with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of active ingredient in the form of a salt with another counter-ion, for example using a suitable ion exchange resin.

Examples of pharmaceutically acceptable addition salts include those derived from mineral acids, such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

The amount of agent (or salt thereof) present in the compositions of the invention may vary considerably according to the intended form of the intraoral product and the intended release profile. The composition may comprise the alkaline active pharmaceutical agent in an amount of from 0.1% to 99% by weight of the composition. In embodiments in which the alkaline active pharmaceutical agent is nicotine or a salt thereof, the composition may, for example, comprise said nicotine or salt thereof in an amount of from 0.1% to 50% by weight.

### Alkaline Bioceramics

As noted above, alkaline bioceramics (i.e. chemically bonded ceramic systems formed from a substance selected from the group consisting of calcium aluminates, calcium silicates, calcium phosphates and mixtures thereof) can provide a stable and sustained source of alkalinity. In one embodiment, the chemically bonded ceramic system is formed from a substance selected from the group consisting of calcium aluminates, calcium silicates, alpha-tricalcium phosphate, tetracalcium phosphate and mixtures thereof.

By the use of the term “chemically bonded ceramic system” (or similar) we refer to materials that are capable of being formed at room temperature or at slightly elevated temperature (e.g. less than about 200° C.). In some embodiments, for example when the alkaline active pharmaceutical agent is nicotine, these systems may also act as carriers in the compositions of the invention as they contain pores within which the alkaline active pharmaceutical agent (or salt thereof) may be located.

The term chemically bonded ceramic” typically refers to a system formed from a self-setting precursor material. Chemically bonded ceramic systems include non-hydrated, partly hydrated or fully hydrated ceramics. Therefore, in an embodiment of the invention, the alkaline bioceramic is based on a ceramic material that is formed from a self-setting precursor ceramic. The precursor substance typically consumes a controlled amount of water to form a network. Precursor substances that may be mentioned in this respect include CaOAl₂O₃, (CaO)₁₂(Al₂O₃)₇, (CaO)₃(Al₂O₃), (CaO)(Al₂O₃)₂, (CaO)₃(SiO₂), (CaO)₂(SiO₂), alpha-tricalcium phosphate, and tetracalcium phosphate (Ca₄(PO₄)₂O). These substances are all capable of reacting with water at room temperature to form an alkaline bioceramic. Their chemistry is known in the art. Water may be provided in liquid form, or the precursor substance may be exposed to a humid atmosphere for a sufficient amount of time for the material to cure and harden.

For example, alpha-tricalcium phosphate (α-TCP) is widely used in bone cement applications due to its conversion into CDHA (calcium deficient hydroxyapatite) and its excellent biological properties. It is also used extensively as a food additive, as an anticaking agent and in health and beauty products. When mixed with water, αTCP reacts to create calcium deficient hydroxyapatite under the creation of a strongly alkaline pH.

The calcium silicate system is a hydraulic cement system that sets and hardens producing an alkaline environment. Principally two different phases can be used that have significant cementing reactions, it is the C₂S and the C₃S phases. The equilibrium pH is about 12. The principle reaction of C₃S is shown below.

2Ca₃SiO₅+7H₂O→3CaO·2SiO₂·4H₂O+3Ca(OH)₂

The end result is a calcium-silicate-hydrate gel and calcium hydroxide. During this reaction hydroxide ions are released resulting in a high pH.

Other suitable systems available are those based on aluminates and silicates, both of which consume a great amount of water. Phases such CA2, CA, C3A and C12A7, and C2S and C3S in crystalline or amorphous state (C═CaO, A=Al₂O₃, SiO₂═S, according to common cement terminology) may be used, which are readily available. The calcium aluminate and/or calcium silicate phases may be used as separate phases or as mixtures of phases. The above-mentioned phases, all in non-hydrated form, act as the binder phase (the cement) in the carrier when hydrated. The liquid(water)-to-cement weight ratio is typically in the region of 0.2 to 0.5, preferably in the region of 0.3 to 0.4.

Where the chemically bonded ceramic system is formed from a calcium phosphate, particular precursor materials of relevance are alpha-tricalcium phosphate and tetracalcium phosphate. Yet further examples of chemically bonded ceramic systems are those formed from a different calcium phosphate or a calcium sulphate. However, these systems do not necessarily act as a source of hydroxide ions so are not considered to be alkaline bioceramics”. These systems are nevertheless useful as carriers of the alkaline active pharmaceutical agent and so are described in more detail elsewhere herein. Suitable calcium sulphate or calcium phosphate starting materials can be used to form chemically bonded ceramic systems using the methods and procedures described hereinbelow in respect of alkaline bioceramics based on calcium aluminate for example.

Once formed, the alkaline bioceramic is usually a porous solid. The water-to-cement ratio during manufacturing, particularly for chemically bonded ceramic systems formed from calcium aluminates and calcium silicates, is important for the pore size and pore volume. As an example, for a phase pure 1:1 CA precursor the theoretic water-to-cement (“W/C”) ratio that gives complete hydration and complete use of all water is about 0.4. If the W/C ratio is increased, any excess water present will result in an increased pore volume and, to some extent, increased pore size. For calcium aluminates and calcium silicates, the permissible range of water content is quite wide, i.e. it is possible to increase the W/C ratio far above the theoretic value needed for complete hydration and still have a hardened body with sufficient structural integrity.

The chemically bonded ceramic system is formed from a suitable precursor material, such as a calcium aluminate, which is typically provided in a powdered form for hydration. The mean grain size of any precursor powder particles may be below about 500 μm, e.g. below about 100 μm, preferably between about 1 μm and about 30 μm. This is to enhance hydration. Such precursor material may be transformed into a nano-size microstructure during hydration (e.g. when brought into contact with liquid water or a humid atmosphere). This reaction involves dissolution of the precursor material and repeated subsequent precipitation of nano-size hydrates in the water (solution) and upon remaining non-hydrated precursor material. This reaction favourably continues until precursor materials have been transformed and/or until a pre-selected porosity determined by partial hydration using the time and temperature, as well as the H₂O in liquid and/or humidity, is measured.

Pore sizes in chemically bonded ceramic systems may be controlled by various techniques during the process of fabricating the carrier material network structure. A particular method that is suitable for use with the chemically bonded ceramic systems used in the present invention is the porogen leaching method which involves the use of a sacrificial phase during the formation of the chemically bonded ceramic system. A porogenic material may be included as part of the reaction mixture during the formation of the chemically bonded ceramic system in order to assist in the formation of pores within the final carrier material network. Porogenic materials include, for example, oils, liquids (e.g. water), sugars, mannitol etc. The porogenic material may then be removed from the carrier, e.g. by burning it away through heating during the curing process, or by dissolving it away using an appropriate solvent. Dissolving is usually achieved with water in order to avoid leaving residual amounts of a substance which may have deleterious effects on the formulation or adverse effects on the user. In such cases, the alkaline active pharmaceutical agent (or salt thereof) may subsequently be loaded into the pores in the chemically bonded ceramic system by any of the methods disclosed elsewhere herein. A porogenic material with a secondary function may also be used. For example, the porogenic material may act as a source of flavour, e.g. as a sweetener. In such cases, it is not essential for the porogenic material to be removed from the carrier prior to use. Instead, some or all of the porogenic material may remain in the carrier, alongside the nicotine (or salt thereof), so that both substances can be released in the user's mouth. Porogenic materials that rapidly dissolve in water are particularly suited for use in this way. Still further, porogenic materials that slowly dissolve in water may be useful for controlling the release of the alkaline active pharmaceutical agent, for example when sustained-release is desired. Porogenic materials that may be used as sweeteners include sweetening agents known in the art, particularly mono-, oligo- and poly-saccharides; sugar alcohols such as mannitol, sorbitol, maltitol and xylitol; natural and synthetic sweeteners such as sucrose, glucose, dextrose, maltose, fructose, saccharin, aspartame, acesulfame K, sucralose, saccharin and cyclamates; and mixtures thereof.

Foaming methods may also be used to increase the pore sizes in chemically bonded ceramic systems. Such methods would be known to the skilled person and are particularly useful for forming carriers with larger pore sizes. One example of such a method involves preparing the carrier using carbonated water or water containing air bubbles. This may also be achieved without the use of foaming agents (e.g. Tween 80).

The total porosity of the chemically bonded ceramic system may be from about 10% to about 70%, such as from about 20% to about 40%. Porosities and average pore sizes may be measured by methods known to the skilled person, for example the mercury intrusion method, the BET (Brunauer, Emmet, and Teller) method, and N₂-adsorption techniques.

The intraoral products and intraoral compositions of the invention may contain other substances that provide a source of alkalinity in the mouth. For example, the products and compositions may contain more than one alkaline bioceramic. They may also contain one or more of the precursors for the alkaline bioceramics disclosed herein (i.e. one or more substances selected from the group consisting of calcium aluminates, calcium silicates, and calcium phosphates) as these substances can act as a source of alkalinity. In another embodiment, the product or composition of the invention may contain sodium carbonate and sodium bicarbonate. However, in a particular embodiment, sodium carbonate and sodium bicarbonate are substantially absent from the composition or product. In a further embodiment, the alkaline bioceramic is the sole source of alkalinity in the composition or product.

In one aspect of the invention, the alkaline bioceramic is used as a carrier for at least a portion of the alkaline active pharmaceutical agent. The porous nature of the alkaline bioceramic makes it potentially suitable for such purposes. In a particular example of such systems, the alkaline active pharmaceutical agent is nicotine or a salt thereof, though the alkaline bioceramic may be used as a carrier for any alkaline active pharmaceutical agent.

The chemically bonded ceramic systems used in the composition of the invention may be loaded with alkaline active pharmaceutical agent (or a salt thereof) by soaking the porous chemically bonded ceramic system in a liquid containing the agent or its salt, or through any other method which facilitates the drawing up of that substance into the pores of the chemically bonded ceramic system via capillary forces (including spraying, brushing, rolling, dip coating, powder coating, misting or vacuum enhanced loading).

However, preferably the alkaline active pharmaceutical agent (or salt thereof) is present when the chemically bonded ceramic system is formed. This allows for greater control over the extent to which the pores in the chemically bonded ceramic system are filled with the agent, and so allows for greater control over the release characteristics of the final formulation. In particular, this loading method enables the total amount of agent that is stored in, and released from, the chemically bonded ceramic system to be more reliably controlled.

In a preferred embodiment of the invention, the agent (or salt thereof) is co-formedly interspersed in pores within the carrier material network. This means that, whatever process is employed to form the carrier, it must also necessarily form pores within which the agent is interspersed. Where the process by which the alkaline bioceramic carrier is formed involves the use of porogens, the agent will also be interspersed within the pores arising directly from the use of said porogens. Chemically bonded ceramic systems are particularly suited for use in such embodiments as the process by which the system and its pore network is formed does not require very high temperatures, in contrast to sintered ceramics.

The agent (or salt thereof) may be mixed with precursor(s) to the chemically bonded ceramic system using a variety of techniques, such as dry powder mixing. Alternatively, the agent (or salt thereof) and precursor(s) may be mixed by way of a sol-gel process, as a solution, or as a slurry, a paste or a putty of, for example, particles, granules or pellets of said precursor(s), in the presence of an appropriate liquid (e.g. an aqueous or organic solvent). This mixing step is followed by some sort of “curing” process to form the chemically bonded ceramic system, which comprises pores within which the agent resides. Chemically bonded ceramic systems that are formed in this way may be said to be pre-loaded with the alkaline active pharmaceutical agent.

The pores formed during the formation of the alkaline bioceramic are themselves a three-dimensional network of channels or voids within the solid network, containing (e.g. particles of) the agent or a salt thereof. Such pores may thus be essentially “secondary pores” formed by chemical interactions (e.g. “bonding”) between the surfaces of primary particles of carrier (which may be porous in their own right (i.e. comprise “primary” pores). Such pores may, for example, result from exposure of such materials to one or more chemical reagents that cause a physical and/or chemical transformation (such as a partial dissolution) at, and subsequent physical and/or chemical bonding together of, those surfaces (which may in itself result as a consequence of some other physico-chemical process such as drying, curing, etc.), giving rise to said pores/voids.

However, such secondary pores are not necessarily formed in this way, and bonding together of primary particles of chemically bonded ceramic material may also be physical and/or mechanical, or may be formed during the production of a three-dimensional, chemically bonded ceramic network as described hereinbefore, in the presence of the agent.

Porous carrier materials typically contain both open pores and closed pores. The term “open pores” refers to pores (e.g. voids within the material) that are open to the external environment such that, when those pores are otherwise empty, gases in the environment are able to pass in and out of those pores. Such pores are generally located at or close to the surface of the individual carrier material particles. The surface of the porous chemically bonded ceramic material will typically be rough due to the porosity of the material. The surface may also contain pits and dents and these are included within the term “open pores”. The term “closed pores” refers to pores which are located within particles of carrier material away from the external surfaces, and which may contain material (e.g. gases) which is not able to freely exchange with the external environment.

Preferably, the precursors for the chemically bonded ceramic system are mixed with the agent (or salt thereof) prior to the hardening process taking place. The agent (e.g. nicotine) is then present at the moment when pore formation occurs with the result that the agent becomes located within the pores of the chemically bonded ceramic system. Therefore, in one embodiment of the composition of the invention, at least a portion of the agent or salt thereof (e.g. the nicotine or salt thereof) is located within pores in the (hardened) chemically bonded ceramic system. By this, we mean that at least 20% (e.g. at least 30%, at least 40%, at least 50%, at least 60%, at least 70%, or at least 80%) by weight of the agent (or salt thereof) is located within pores in the chemically bonded ceramic system. In one embodiment, essentially all of the agent (or salt thereof) is located within the pores of the alkaline bioceramic. By this, we mean that at least 90% (e.g. at least 95%, preferably at least 98%) by weight of the agent (or salt thereof) is located within the pores of the alkaline bioceramic. For the avoidance of doubt, these features apply equally to compositions in which a different chemically bonded ceramic system, e.g. a system formed from calcium sulphate as described elsewhere herein, is used as a carrier for the agent or salt thereof.

In embodiments in which the chemically bonded ceramic system is loaded with alkaline active pharmaceutical agent (or a salt thereof), the composition of the invention may also contain the following:
(i) an additional amount of alkaline active pharmaceutical agent (e.g. nicotine) or salt thereof that is not loaded into pores in the chemically bonded ceramic system; and/or
(ii) an additional amount of a substance selected from the group consisting of calcium aluminates, calcium silicates, calcium phosphates and mixtures thereof.

In the case of (ii) above, these substances are precursors to the chemically bonded ceramic system and are also capable of acting as a source of alkalinity.

It is not essential that the alkaline bioceramic be used as a carrier of the alkaline active pharmaceutical agent. The agent may be incorporated into the composition using a different carrier or without any carrier at all.

For example, the alkaline active pharmaceutical agent (e.g. nicotine) may be bound to, adsorbed to, absorbed into, enclosed into or form a complex or any other non-covalent bond with one or more other components, such as starch, alginate salts, beta-cyclodextrin and cellulose. International patent application no. WO 2010/104464 A1 describes a matrix of alginate salt which entraps a biological active substance, such as nicotine within the matrix. US patent publication no. US 2005/0053665 A1 describes nicotine which is absorbed into or adsorbed to cellulose of non-seed origin. International patent application nos. WO 2007/104573 and WO 2010/031552 relate to a nicotine-cellulose combination for the preparation of a nicotine pouch composition. The disclosures in these documents are hereby incorporated by reference. Examples of different carriers that may be used in the present invention include microcrystalline cellulose, hydroxypropyl methylcellulose, and other materials that are conventionally known in the art as a carrier for nicotine in nicotine pouches.

In a particular example, the agent is incorporated into the composition using a cellulose carrier. Said cellulose may comprises internal voids and/or pores which at least partially comprise nicotine. In an embodiment, said cellulose carrier preferably has a surface area of at least 0.7 m²/g. Suitable celluloses include those derived from a plant, an algae, a bacterium, a fungi, or combinations thereof. Microcrystalline cellulose is particularly preferred. Commercially available materials that may be used include AVICEL(R) grades PH-100, PH-102, PH-103, PH-105, PH-112, PH-113, PH-200, PH-300, PH-302, VIVAPUR(R) grades 101, 102, 12, 20 and EMCOCEL(R) grades 50M and 90M, and mixtures thereof.

A preferred example of a carrier that may be used in the compositions of the invention is a chemically bonded ceramic system formed from a calcium sulphate or a calcium phosphate, particularly a neutral or acidic calcium sulphate or a neutral or acidic calcium phosphate. Particular examples of such substances include alfa or beta phase calcium sulphate hemihydrate (end product calcium sulphate dihydrate), neutral calcium phosphate (apatite) and acidic calcium phosphate (brushite). Beta tricalcium phosphate is a further example. Chemically bonded ceramic systems formed from these calcium sulphate or calcium phosphate precursors do not typically act as a source of alkalinity. These materials can be used to form chemically bonded ceramic systems using the methods and procedures described hereinbefore, for example, in respect of calcium aluminate.

We have found that it is difficult to the form an intraoral composition containing a pH regulating agent alongside a chemically bonded ceramic system loaded with an adequate amount of nicotine. If a source of alkalinity (besides the nicotine) is present during the process of forming the hardened porous chemically bonded ceramic system, a significant proportion of the nicotine can be lost. Without wishing to be bound by theory, it is believed that the primary mechanism for loss is evaporation of nicotine in the un-ionised (i.e. non-protonated) form. It is also believe that similar loss would occur for other alkaline active pharmaceutical agents. We have surprisingly found that this evaporative loss can be greatly reduced or essentially completely eliminated by allowing the chemically bonded ceramic system used as the carrier for the agent to be loaded with the agent in the absence of any (other) source of alkalinity. Once the hardened carrier loaded with agent has been formed, the alkaline component (i.e. the alkaline bioceramic) can then be introduced.

By way example only, an intraoral composition of the invention comprising nicotine as the alkaline active pharmaceutical agent may be formed by a process comprising:
(i) forming a solid, porous chemically bonded ceramic system using calcium sulphate in the presence of nicotine (or a salt thereof);
(ii) optionally processing the loaded, hardened chemically bonded ceramic system (e.g. by grinding into a powder); and
(iii) mixing the product of step (i) or (ii) with an alkaline bioceramic.

Thus, in one embodiment, the invention relates to an intraoral composition comprising the following components:
(a) an alkaline active pharmaceutical agent or a salt thereof;
(b) a solid, porous chemically bonded ceramic system formed from a calcium sulphate or a calcium phosphate (e.g. a neutral or acidic calcium sulphate or calcium phosphate); and
(c) an alkaline chemically bonded ceramic system formed from a substance selected from the group consisting of calcium aluminates, calcium silicates, calcium phosphates and mixtures thereof.

Preferably, in such embodiments, component (b) is formed before it is introduced to either component (c) or the (inorganic) precursors to component (c).

According a further aspect of the invention, there is provided a method of forming an intraoral composition of the invention, wherein the method comprises bringing an alkaline active pharmaceutical agent or salt thereof into association with a chemically bonded ceramic system formed from a substance selected from the group consisting of calcium aluminates, calcium silicates, calcium phosphates and mixtures thereof. In one embodiment, the method involves the step of hydrating the calcium aluminate, calcium silicate, or calcium phosphate (or mixture thereof) in the presence of the alkaline active pharmaceutical agent to form a chemically bonded ceramic system. In a separate embodiment, the method involves the step of hydrating a calcium sulphate or a calcium phosphate (e.g. a neutral or acidic calcium sulphate or a neutral or acidic calcium phosphate) in the presence of the alkaline active pharmaceutical agent before it is brought into association with chemically bonded ceramic system formed from a substance selected from the group consisting of calcium aluminates, calcium silicates, calcium phosphates and mixtures thereof

The agent or salt thereof (i.e. component (a)) may be present during the curing phase of component (b), i.e. the agent can be intermixed with the precursor materials for the chemically bonded ceramic system of component (b) prior to hardening, with the result that good control of the loading of the agent can be achieved. As is hereinbefore described, component (b) may alternatively be loaded with the agent (or salt thereof) after the chemically bonded ceramic mass has been formed, e.g. by soaking the ceramic material in a liquid containing the agent or its salt, or through any other method which facilitates the drawing up of that substance into the pores of the ceramic material via capillary forces (including spraying, brushing, rolling, dip coating, powder coating, misting vacuum enhanced loading).

Embodiments which comprise components (a) to (c) above may be used with any agent but are particularly suited for use with opioid analgesics. Furthermore, in such embodiments, a particular precursor material for component (b) that may be mentioned is a calcium sulphate, e.g. alfa or beta phase calcium sulphate hemihydrate. In particularly preferred intraoral compositions, the chemically bonded ceramic system of component (b) is based on a calcium sulphate and is formed in the presence of the agent or salt thereof (e.g. an opioid analgesic such as fentanyl or buprenorphine.)

For the avoidance of doubt, it is not essential for all of the agent (e.g. nicotine or opioid analgesic) in the formulation to be located within the pores of any of the chemically bonded ceramic systems. Some of the agent in the intraoral composition may be located outside of such pores. For example, where the intraoral product is provided in the form a nicotine pouch containing the other components of the powdered intraoral composition, a portion of the nicotine (preferably a powdered salt of nicotine) may be mixed with the other powdered components (which may, in turn, include a solid, porous chemically bonded ceramic system comprising pores loaded with an amount of nicotine or salt thereof) prior to being placed in the pouch. Said mixing may be achieved by spraying a nicotine solution onto particles of the nicotine-loaded chemically bonded ceramic system, and then allowing that mixture to dry prior to being placed in the pouch. In such embodiments, the amount of nicotine (or salt thereof) that is added to the hardened, powdered chemically bonded ceramic system is from 1 to 50%, from 5 to 40% or from 10 to 30% by weight of the total amount of nicotine (or salt thereof) in the intraoral product.

In a further example, where the final intraoral product is provided in the form of a nicotine pouch containing either the powdered formulation or a combination of a solid, porous chemically bonded ceramic system and nicotine (or a salt thereof), a portion of the nicotine may be associated with the material that forms the pouch, i.e. a portion of the nicotine is not located within cavity of the bag (as is described elsewhere herein) but is instead bound to or incorporated within the walls of the bag itself. This may be achieved e.g. by soaking the pouch material in a solution containing nicotine (as base or dissolved nicotine salt) and then allowing the solvent (e.g. water or another appropriate solvent) to evaporate leaving nicotine or a salt thereof incorporated into the pouch material. In embodiments the intraoral product is a nicotine pouch and a portion of the nicotine in said intraoral product is associated with the material that forms the pouch, the amount of nicotine (or salt thereof) associated with the material may be from 0.1 to 30%, from 0.5 to 20% or from 1 to 10% by weight of the total amount of nicotine (or salt thereof) in the intraoral product.

Typically, once a hardened mass of chemically bonded ceramic component (b) (e.g. a hardened mass of chemically bonded ceramic system which has been loaded with nicotine or a salt thereof) is formed, it is then ground to form a powder which is subsequently mixed with alkaline bioceramic (component (c)). The grinding process may also be performed in the presence of the alkaline bioceramic to ensure efficient mixing.

As the alkaline bioceramic is not present during the formation of component (b), the alkaline bioceramic does not become incorporated into the pores of component (b). Ingress of a small amount of the alkaline bioceramic into the pores of component (b) may occur when the two components are brought together or during subsequent processing, but only minimal ingress will occur in this way. Therefore, in one embodiment, the alkaline bioceramic is predominantly located outside of the pores of the chemically bonded ceramic system of component (b). By this, we mean that at least 90% (e.g. at least 95%, preferably at least 98%) by weight of the alkaline bioceramic is located outside of the pores of the chemically bonded ceramic system of component (b).

For the avoidance of doubt, it is not essential that any pores in the chemically bonded ceramic system of the alkaline bioceramic contain any of the agent or salt thereof. The alkaline bioceramic system may be formed (e.g. through hardening a hydrated mass of the appropriate calcium salt) before it is added to the other components of the composition of the invention.

### Intraoral Products

The invention relates to intraoral products that are intended to release an alkaline active pharmaceutical agents (or a salt thereof) upon exposure to moisture in the mouth. Where the term “oral product” is used herein, it refers to an intraoral product that is intended to administer the alkaline active pharmaceutical agent transmucosally. The products are capable of both immediate and sustained release, but are particularly suited when slow and/or sustained release is desired.

Oral products that are capable of providing a sustained release of agent allow the user to obtain a long-lasting sensory experience or therapeutic exposure using a minimal number of products per day. Where the product is intended to be used as part of a therapy (e.g. of treating nicotine dependence), this characteristic improves patient compliance and minimises interference with the individual's lifestyle.

The term “sustained-release” is employed herein synonymously with the term “controlled-release”, and will be understood by the skilled person to include intraoral products that provide, and/or are adapted to provide, for a “sustained”, a “prolonged” and/or an “extended” release of agent (in which agent is released at a sufficiently retarded rate to produce a therapeutic response or give a pleasurable experience over an extended period of time compared to intraoral products currently on the market).

Intraoral products containing the intraoral compositions of the invention are capable of achieving a nearly constant rate of release over an interval of from about 10 minutes to about 1 hour, potentially longer. Constant release over longer periods is particularly desirable for medicinal products. In one embodiment, the intraoral products are capable of achieving a nearly constant rate of release over an interval of from about 10 to 30 minutes. What is meant by this is that nicotine release from the product continually occurs (at a non-zero rate) over the specified time interval. Constant release may further be defined as a composition being capable of maintaining a steady state concentration of agent (or a metabolite thereof) in a body fluid not deviating more than about 20% (e.g. about 10%) from the mean value during the dose interval.

The intraoral compositions of the invention, when held in the mouth, are advantageously capable of releasing a high proportion of the material held in the pores. By this we mean that, when the intraoral composition is held in the mouth for an extended period of time (e.g. at least 10 minutes or preferably at least 20 minutes), at least 75% (e.g. at least 80%, at least 85% or at least 90%) of the alkaline active pharmaceutical agent stored in the pores of the chemically bonded ceramic system is released from said pores and made available to the user. This characteristic of near-complete release allows the user to know more accurately how much active ingredient is taken into the body, which may be advantageous when dosing a patient for therapeutic purposes. When the intraoral compositions of the invention are used in nicotine pouches, such as those described herein where nicotine is held in pores in a chemically bonded ceramic system, once the nicotine (or salt thereof) has been released from those pores it is immediately able to escape the pouch and be taken up by the user.

In addition, the intraoral compositions of the invention are capable of achieving essentially complete release in a timeframe suitable for therapeutic use. As is demonstrated in the Examples, the intraoral compositions of the invention are capable of releasing almost all of the releasable alkaline active pharmaceutical agent in around 20 minutes when held in the mouth. This is particularly relevant for nicotine products because users of nicotine pouches typically hold an individual pouch in their mouth for a period of from five minutes to one hour whilst the nicotine is released, with 20 to 30 minutes being the most common timeframe. This characteristic helps to minimise wastage of unused nicotine when a product has been consumed, and makes the intraoral compositions of the invention, and pouches containing said intraoral compositions, particularly well-suited to use in therapeutic treatment, such as nicotine replacement therapy or aiding smoking cessation.

The dry formulations of the invention have also been found to provide rapid release of the water soluble components. Studies involving human volunteers found that the onset of sensations associated with flavours was more rapid for the dry formulations of the invention compared with dry commercial formulations. The time required for onset of nicotine sensation for the dry formulations of the invention was equal to or less than that for dry commercial formulations. The formulations of the invention are therefore capable of generating a rapid sensory experience for the user.

The total amount of nicotine that is delivered by individual nicotine-containing intraoral products is preferably from about 0.5 mg to about 15 mg, e.g., from about 1 mg to about 10 mg. For example, the product may deliver from about 1 mg to about 8 mg, from about 1.5 mg to about 7.5 mg, from about 2 mg to about 5 mg, from about 2.5 mg to about 5 mg, from about 3 to about 10 mg, from about 3 to about 7.5 mg or from about 3 mg to about 5 mg. In a further example, the product may contain about 1.5 mg, about 2 mg, about 2.5 mg, about 3 mg, about 3.5 mg, about 4 mg, about 5 mg or about 6 mg, as calculated as free nicotine base. In particular the amount of nicotine is 2 mg, 3 mg, 4 mg or 6 mg. The above values refer to the amount of nicotine in the free base form that can be delivered from each intraoral product, irrespective of whether the nicotine in the product is provided as free base, a salt or in any other form.

The intraoral compositions of the invention are suitable for use in conventionally sizes nicotine pouches that are intended to be used by holding the pouch in the mouth under the lip. Typically, such pouches are rectangular and have a length of about 25 to about 35 mm, and a width of about 10 to about 15 mm.

The intraoral compositions of the invention may also provide a relatively concentrated form of nicotine, thus enabling the manufacture of small pouches that still contain a conventional amount of nicotine and have advantageous release properties as described herein. Intraoral compositions of the invention may contain at least 50 mg (such as least 60 mg, at least 70 mg or at least 80 mg) nicotine (calculated as free base irrespective of the form in which it is present) per gram of composition. By way of an example, 12 mg of nicotine (calculated as free base) may be incorporated into a chemically bonded ceramic system to give a composition with a total mass of no more than 0.15 g. In this context, small pouches may have no dimension larger than 20 mm, preferably no dimension larger than 15 mm. Typically, small pouches are rectangular and have a length of about 10 to about 15 m, and a width of about 8 to about 10 mm.

The amount of nicotine contained within the intraoral product prior to use may exceed the amount of nicotine that is intended to be delivered to the user. This is because a proportion of the nicotine may be trapped within the intraoral product (e.g. within pores of a chemically bonded ceramic system in the intraoral composition) in such a way that complete release of the nicotine is not possible within the likely time period of use. However, preferably the intraoral product (or intraoral composition) is capable of releasing substantially all of the nicotine or salt thereof upon contact with an aqueous liquid (e.g. saliva). By this, we mean that, upon contact with an aqueous liquid, the intraoral product (or intraoral composition) is capable of releasing at least 80% (e.g. at least 90%) by weight of the nicotine or salt thereof.

The intraoral composition may contain any amount of the alkaline bioceramic that is sufficient to provide the desired pH when in use. Typically, the amount present in the composition is chosen to be sufficient to raise the pH of the saliva inside and close to the composition in use to at least 8. In one embodiment of the invention, the alkaline bioceramic is present at from about 0.1% to about 95%, e.g. from about 0.1% to about 50%, by weight of the intraoral composition. The intraoral products described herein (e.g. pouches and sublingual and buccal tablets containing, for example, nicotine or an opioid analgesic) typically have a mass not exceeding 2 grams. In an embodiment of the invention, the alkaline bioceramic is present at from about 1% to about 25% (e.g. from about 3% to about 20%) by weight of the intraoral product (e.g. pouches and sublingual and buccal tablets).

The amount of alkaline bioceramic present in the intraoral composition may differ depending on whether the alkaline bioceramic is present primarily to provide the desired pH when in use, or whether it is also acting as a carrier for the alkaline active pharmaceutical agent. Only a small amount of alkaline bioceramic may be required if the substance is present primarily to provide the desired pH when in use, for example the amount of alkaline bioceramic in the intraoral composition may be from 0.1% to 20% (e.g. from 1% to 10%) by weight of the intraoral composition. In contrast, larger amounts are typically required when the alkaline bioceramic acts as a carrier in addition to providing a pH effect, for example the amount of alkaline bioceramic in the intraoral composition may be from 10% to 98% (e.g. from 50% to 97%, from 60% to 95%, from 80% to 95%,or from 85% to 95%) by weight of the intraoral composition.

In embodiments in which the alkaline bioceramic is formed from a calcium silicate, the amount of alkaline bioceramic in the intraoral composition may be at least 5%, e.g. at least 50% by weight of the intraoral composition. In a particular embodiment, said amount is at least 75%, e.g. 75 to 95%, by weight of the intraoral composition.

The intraoral products may be supplied to users in the form of pouches (similar to snus) and tablets. The term “pouch” as used herein refers to a pouch or bag that is fully or partially loaded with a combination of a solid, porous chemically bonded ceramic system and an alkaline active pharmaceutical agent. Typically the agent is nicotine or a salt thereof. In one embodiment, the pouch also contains an alkaline bioceramic; in such cases the pouch or bag may be said to be fully or partially loaded with an intraoral composition of the invention. In another embodiment, alkaline bioceramic is incorporated into (e.g. bound to) the material that forms the pouch or bag. Nicotine pouches which comprise an alkaline bioceramic both within the cavity of the pouch or bag and incorporated into the material that forms said pouch or bag are also contemplated.

The intraoral products of the invention are particularly suited for transmucosal administration whereby the product (e.g. in the form of a tablet, wafer, lozenge or similar product) containing the composition is placed in contact with the lip, gum or cheek for an extended period (several minutes) while the agent is released. The intraoral products may be supplied to the end user in bulk. That is, the present invention also relates to a package containing a plurality of intraoral products (e.g. at least two pouches or tablets containing a composition of the invention).

Pouches (e.g. snus pouches) containing the intraoral compositions of the invention in which the agent is nicotine (or a salt thereof) may also contain ground tobacco. However, in particular embodiments, these intraoral products do not contain ground tobacco and may therefore be described as a tobacco-free version of snus. In such systems, the pouch or bag contains a specific amount of the intraoral composition, and thereby a specific amount of nicotine, but typically does not include any tobacco dust, leaf or stem.

When the agent (or salt thereof) in the intraoral product is incorporated into the pores of a chemically bonded ceramic system, it is believed to have an enhanced stability profile compared to commercially available formulations, i.e. compared to formulations in which the agent is not stored within pores of a chemically bonded ceramic carrier of the sort disclosed here. Without wishing to be bound by theory, it is believed that incorporation of an agent susceptible to oxidation (such as nicotine) into the pores of a chemically bonded ceramic system prevents or slows the oxidative degradation, thus extending the shelf-life of the product.

An embodiment of the present invention concerns a pouch (e.g. a nicotine pouch) comprising a permeable, sealed bag containing the agent, or a salt thereof, and an alkaline bioceramic. The bag is typically made of a permeable material that encloses a cavity. The powdered intraoral composition is held within the cavity, but soluble components of the composition are able to pass through the bag material when the bag is exposed to water (e.g. saliva). Suitable materials for pouches are known to the skilled person, and include paper of the sort used in tea bags, filter paper, and the like. Other materials include heat-sealable non-woven cellulose, such as long fiber paper, cotton and silk.

The intraoral product of the invention also provides stable storage of the agent (e.g. nicotine) or salt thereof prior to use. The product of the invention is preferably stored in an airtight container, such as a tin or bag, prior to use, and it may be stored in this way for several weeks or months (e.g. up to at least one year) without significant loss of the agent. Suitable storage containers are known to the skilled person and include any conventional closable container. These storage containers may provide a convenient and portable system capable of holding multiple pouches or tablets. A further aspect of the invention therefore relates to a closable container comprising one or more, and preferably a plurality of, intraoral products of the invention (e.g. pouches, sublingual tablets or buccal tablets as described herein).

As is hereinbefore mentioned, the agent (e.g. nicotine) may be incorporated into pores of a chemically bonded ceramic system that is not an alkaline bioceramic. The alkaline bioceramic may therefore be incorporated into the final product through other methods, e.g. by mixing the alkaline bioceramic with the hardened chemically bonded ceramic system as is hereinbefore described. Alternatively, or additionally, the alkaline bioceramic may be associated with the bag material itself. For example, particles of the alkaline bioceramic may be embedded within the permeable material of the bag so that the alkaline bioceramic is readily able to release hydroxide ions upon contact with saliva. Thus, in one embodiment, the bag comprises a cavity surrounded by a permeable material, and the alkaline bioceramic is associated with the permeable material. Other suitable methods of incorporating the alkaline bioceramic into the bag material would be known to the skilled person.

In embodiments of the invention, particularly those involving nicotine, the intraoral composition is contained within a pouch, and the total weight of the loaded pouch is from 0.2 to 3 g, such as from 0.4 to 2 g. In another embodiment, the pouch has a weight and/or volume similar to commercially available portion snus products and nicotine pouches. In a further embodiment of the invention, the intraoral composition is contained within a small pouch having a total weight of from 0.1 to 0.5 g, such as from 0.1 to 0.4 g. Small pouches are discussed elsewhere herein and the utility of such low weight products is possible thanks to the fact that the chemically bonded ceramic systems referred to herein are capable of providing a very compact and stable storage of agents such as nicotine whilst maintaining a suitable release profile.

Pouches, such as nicotine pouches, may be manufactured using methods known to those skilled in the art, particularly those methods used for the manufacture of snus and commercially available nicotine pouches (such as ZYN®). For example, the powdered contents for the pouch may be made using the methods described herein or using conventional methods known in the art, and the powder may then be then loaded into sealable bags, e.g. heat-sealable bags. Such bags should be water insoluble and permeable to saliva. Suitable materials for pouches are described hereinbefore, and are also known to the skilled person, for example from U.S. Pat. No. 9,161,908. Heat-sealable non-woven cellulose, such as long fiber paper, offers a particularly suitable material for use in nicotine pouches. Once a prescribed amount of the powder is filled into the pouch, it is maintained in the pouch by sealing. Uptake of agent (e.g. nicotine) in the mouth may be facilitated by incorporating a bioadhesion and/or mucoadhesion promoting agent into the pouch. The bioadhesion and/or mucoadhesion promoting agent may be provided in cavity of the bag. Alternatively, or additionally, that agent may be incorporated into or combined with the bag material.

Tablet-based intraoral products that may be mentioned include sublingual tablets, buccal tablets, wafers and lozenges. In this form, the intraoral product containing the intraoral composition of the invention is intended to be placed under tongue, under the lip, against the gum, or against the cheek, and the agent (e.g. opioid analgesic) is absorbed through the surrounding mucous membranes. References to “sublingual tablets” elsewhere herein include references to buccal tablets except where indicated otherwise. Adhesion to the interior surface of the mouth may be facilitated by incorporating a bioadhesion and/or mucoadhesion promoting agent into the tablet, wafer or lozenge.

The bioadhesion and/or mucoadhesion promoting agent is effective in making the tablet or pouch adhere to the oral mucosa and may, in addition, possess properties to swell and expand in contact with water and thus make a tablet disintegrate when wetted with saliva.

The expression “mucoadhesion” is meant to denote an adhesion to mucous membranes which are covered by mucus, such as those in the oral cavity, while the expression “bioadhesion” is meant to denote an adhesion to biological surfaces more in general, including mucous membranes which are not covered by mucus. These expressions generally overlap as definitions, and may usually be used interchangeably, although the expression “bioadhesive” has a somewhat wider scope. In the present specification and claims, the two expressions serve the same purpose as regards the objects of the invention, and this has been expressed by the use of the common term “bio/mucoadhesion”. Suitably the tablet contains from 0.1 up to 25 weight percent of bio/mucoadhesion promoting compound, based on the total weight of the tablet.

It is preferred that the bio/mucoadhesion promoting agent is a polymeric substance, preferably a substance with an average molecular weight above 5,000 Daltons (weight average). A variety of polymers known in the art can be used as bio/mucoadhesion promoting agents. Examples of such bio/mucoadhesion promoting agents include cellulose derivatives such as hydroxypropylmethyl cellulose (HPMC), hydroxyethyl cellulose (HEC), hydroxypropyl cellulose (HPC), methyl cellulose, ethyl hydroxyethyl cellulose, carboxymethyl cellulose, modified cellulose gum and sodium carboxymethyl cellulose (NaCMC); starch derivatives such as moderately cross-linked starch, modified starch and sodium starch glycolate; acrylic polymers such as carbomer and its derivatives (Polycarbophyl®, Carbopol®), etc.); polyvinylpyrrolidone; polyethylene oxide (PEO); chitosan (poly-(D-glucosamine)); natural polymers such as gelatin, sodium alginate, and pectin; scleroglucan; xanthan gum; guar gum; poly co-(methylvinyl ether/maleic anhydride); microcrystalline cellulose (Avicel®); and croscarmellose (e.g. crosscarmellose sodium). Such polymers may be crosslinked. Combinations of two or more bio/mucoadhesive polymers can also be used. Substances like HPMC that can produce a smooth surface for the finished tablet whilst still enabling the tablet to stick to oral mucosa are particularly advantageous.

In an embodiment of the invention, the intraoral product is a tablet having a total weight of from 25 to 200 mg, such as from 50 to 150 mg.

Tablets (e.g. sublingual tablets) may be manufactured using methods known to those skilled in the art, such as dry powder blending, granulation and tablet pressing. Tablets may also be made by mixing the dry ingredients, including the precursor(s) for the chemically bonded ceramic system, adding water, wet mixing and then casting. The final tablet may, for example, be a buccal tablet that does not fully dissolve in the mouth but which needs to be physically removed (e.g. swallowed) once used. The tablet may alternatively be a dissolving tablet in which all components are dissolved and either absorbed or swallowed.

Pouches and tablets may also contain one or more other ingredients selected from the group consisting of a filler (typically a food grade filler), water, salt, and flavouring. It is not essential that any of these other ingredients is incorporated into the pores of the chemically bonded ceramic system(s), however any such ingredients that are incorporated in this way and are water soluble will be expected to have a similar release profile to the agent contained within the composition.

Fillers that increase saliva production may also advantageously be used in the products described herein, particularly nicotine-containing products. Such substances, sometimes known as sialogogues, are known in the art and include salt (NaCl), sweeteners (e.g. xylitol) and acidic substances (such as malic acid and ascorbic acid). The agent in the intraoral product is able to dissolve in saliva and is then be absorbed through the oral mucosa. Flavourings in the products are also transported by saliva to taste buds in the oral cavity. The incorporation of a substance that increases saliva production improves the experience for the user by ensuring that the sensations arising from the agent (e.g. nicotine) and flavourings in the product can occur more quickly.

Other fillers that may be used in pouches include rubber arabicum, microcrystalline cellulose, maltitol. Yet more fillers that may be mentioned include inert inorganic fillers such as alumina, zirconia, and glass.

Fillers that may be used in sublingual tablets (and wafers, lozenges, etc.) include conventional fillers known to the skilled person in the context of pharmaceutical formulations, particularly oral tablets and capsules. Appropriate materials are well known to the person skilled in the art; see, for instance: Dosage Forms: Tablets. Volume 1, 2nd Edition, Lieberman H A et al. New York and Basel 1989, p. 354-356, and literature cited therein.

Fillers are typically present in the intraoral product in an amount ranging from about 10% to about 90% by weight of the intraoral product.

Controlled release agents may also be incorporated into the product. Such agents slow the rate of release of agent from the product and thereby extend the duration of the sensory experience for the user. It is preferred that the controlled-release agent is a material that is capable of providing a sustained-release, a delayed-release or both.

In this respect, it is preferred that the controlled-release agent is a polymer. Examples of polymers that may be employed as controlled-release agents include, without limitation: alkylcellulose polymers (e.g. ethylcellulose polymers), and acrylic polymers (e.g. acrylic acid and methacrylic acid copolymers, methacrylic acid copolymers, methyl methacrylate copolymers, ethoxyethyl methacrylates, cyanoethyl methacrylate, methyl methacrylate, copolymers, methacrylic acid copolymers, methyl methacrylate copolymers, methyl methacrylate copolymers, methacrylate copolymers, methacrylic acid copolymer, aminoalkyl methacrylate copolymer, methacrylic acid copolymers, methyl methacrylate copolymers, poly(acrylic acid), poly(methacrylic acid, methacrylic acid alkylamid copolymer, poly(methyl methacryate), poly(methacrylic acid) (anhydride), methyl methacrylate, polymethacrylate, methyl methacrylate copolymer, poly(methyl methacrylate), poly(methyl methacrylate) copolymer, polyacryamide, aminoalkyl methacrylate copolymer, poly(methacrylic acid anhydride), and glycidyl methacrylate copolymers). The polymer may also be a mixture of polymers. Typically, the molecular weight (weight average and/or number average) of the polymer is 1,000 to 10,000,000, 10,000 to 1,000,000, preferably 50,000 to 500,000 g/mol, as measured by gel permeation chromatography.

Preferred polymers include the alkyl cellulose polymers and acrylic polymers described herein.

In order to improve the sensory properties of the intraoral product of the invention, one or more sweeteners or texture improvers may be added. These substances include sugars such as sucrose, glucose, dextrose, maltose and/or fructose, sugar alcohols such as mannitol, xylitol, sorbitol, maltitol and/or isomalt, or artificial sweeteners such as sucralose, cyclamates, aspartame, acesulfame and/or saccharin. Sweeteners are particular useful for masking the taste of nicotine in an intraoral product.

Other flavourings that may be used, particularly in nicotine pouches, include menthol, peppermint, wintergreen, sweet mint, spearmint, vanillin, chocolate, black cherry, coffee, cinnamon, clove, tobacco, citrus, fruit flavour and mixtures thereof. In one embodiment, the flavouring is not tobacco. The formulations of the invention may be used with any flavour or combination of flavours. Such flavour may be present in an amount of at least 0.1%, and preferably not more than 5%, by weight of the pouch or tablet.

Substances that give a cooling mouthfeel, such as sugar alcohols (e.g. erythritol), may be used in the intraoral products disclosed herein.

The nicotine pouches described herein may also contain a moist intraoral composition. A moist intraoral composition is an intraoral composition of the invention which additionally contains a component which is either liquid under ambient conditions or is a low-melting solid. Suitable components are known in the art, and include glycerol, propylene glycol (“PG”, E1520), polyethylene glycol (PEG) and sodium alginate (E401). The addition of a relatively small amount of such components advantageously binds the smallest particles together to minimise the formation of fine dust. Fine dust can travel through, or block pores in, the sort of non-woven fabrics typically used in the manufacture of nicotine pouches. Typically, the liquid or low-melting solid is added after the powder has been formed (e.g. by crushing a larger solid mass), and optionally after coarse particles of the chemically bonded ceramic system have been removed. The skilled person would be able to determine the amount of liquid or low-melting solid that needs to be added to the composition of the invention. A suitable amount may range from 0.1% to 10% (such as 2 to 5%) by weight of the composition. It is preferred that the amount is sufficiently low to yield a powdery material that is easily manufactured, handled, transported, divided and/or processed.

Moist pouches, i.e. pouches that contain a moist intraoral composition, generally feel more comfortable in the mouth, and typically provide for quicker initial release of nicotine compared to dry pouches. However, we have found that moist intraoral compositions of the present invention also have surprising sustained release characteristics, similar to the dry formulations. That is, the moist formulations are capable of releasing nicotine (or a salt thereof), as well as other flavours over a sustained period of time. The use of PG, or any other moistening agents of the sorts mentioned hereinabove could also advantageously help prevent unwanted release of material from the pouch, e.g. by agglomerating the finer parts of the powder which may otherwise be able to traverse through the pores in the pouch membrane.

A moist intraoral composition typically has the form of a dough or paste. Such formulations may be made by simple mixing of the liquid or low-melting component with a dry intraoral composition of the invention.

Oral formulations of the invention may also contain one or more preservatives. Substances that may be used as preservatives include sodium benzoate and potassium sorbate as well as other substances known in the art to have similar functionality. Preservatives may be particularly suitable for use in moist intraoral formulations, e.g. those which contain glycerol and/or propylene glycol.

Particular intraoral compositions that may be mentioned include intraoral compositions comprising the following components:
(a) nicotine, an opioid analgesic, or a salt thereof;
(b) a solid, porous chemically bonded ceramic system formed from a calcium sulphate and is formed in the presence of component (a); and
(c) an alkaline chemically bonded ceramic system formed from a substance selected from the group consisting of calcium aluminates, calcium silicates, calcium phosphates and mixtures thereof;
wherein component (b) is formed in the absence of either component (c) (or the inorganic precursors to component (c)). Said intraoral compositions may be advantageously provided in the form of a sublingual or buccal tablet (such as a tablet containing a bioadhesion and/or mucoadhesion promoting agent) or a pouch (i.e. a permeable, sealed bag).

### Medical and Recreational Uses

The intraoral compositions of the invention have medical and/or recreational use.

The intraoral compositions may be placed in the mouth whereupon they are moistened through contact with saliva. The composition may, for example, be placed in the mouth in contact with the lip, gum or cheek, and left there for an extended period. Typically, the composition is retained in the mouth for a period of from five minutes to one hour. Longer durations are possible if the product is tailored to provide slower sustained release of the agent, e.g. by including a controlled release agent such as hydroxypropyl methyl cellulose.

Ingress of moisture into the pores of the chemically bonded ceramic system results in the release of the agent (or salt thereof) contained within. The agent is then taken up into the body at least in part through absorption via the oral mucosa. In an embodiment of the invention, at least 50% by weight of the agent released from the formulation is absorbed through the oral mucosa.

In a medical context, the intraoral composition containing nicotine may be used in the treatment of nicotine dependence, (e.g. nicotine addiction) with a view to aiding an individual in reducing smoking or stopping altogether. The compositions of the invention may therefore be described as being useful in aiding smoking cessation. The intraoral compositions of the invention have several benefits that make them particularly suited to aiding smoking cessation. Firstly, they are capable of releasing almost all of the nicotine stored in the pores, and this in turn allows a user or a clinician to know accurately how much nicotine is being administered to the patient. Secondly, the intraoral compositions provide essentially complete release in a timeframe suitable for therapeutic use, such as within 20 minutes or within 30 minutes. This timeframe is consistent with the amount of time that commercially available nicotine pouches are typically held in the mouth when used. Wastage of nicotine is also minimised as there is very little unused nicotine once a product has been used.

In a further aspect of the invention there is provided a method of treating nicotine dependence (e.g. nicotine addiction) wherein the method involves administration of an intraoral composition of the present invention containing nicotine or a salt thereof to a person suffering from symptoms of nicotine dependence. Similarly, such compositions may be useful in treating (e.g. alleviating) the symptoms of nicotine dependence (including nicotine addiction or nicotine withdrawal), or aiding smoking cessation. Such symptoms may include cravings for nicotine, anger/irritability, anxiety, depression, impatience, trouble sleeping, restlessness, hunger or weight gain, and/or difficulty concentrating. Said use may also be referred to as nicotine replacement therapy.

Nicotine may also be used to ameliorate symptoms associated with various diseases, including dementia, Alzheimer's disease, Parkinson's disease, Huntington's disease, and depression. Thus, in a yet further aspect of the invention there is provided a method of ameliorating symptoms associated with dementia, Alzheimer's disease, Parkinson's disease, Huntington's disease, and depression, wherein the method involves administering an intraoral composition of the invention containing nicotine or a salt thereof to a person suffering from said symptoms.

Compositions the invention are capable of releasing a pharmacologically effective amount of agent during normal use. By “pharmacologically effective amount”, we refer to an amount of agent which is capable of conferring a desired therapeutic effect on a treated patient, whether administered alone or in combination with another active ingredient. Such an effect may be objective (i.e. measurable by some test or marker) or subjective (i.e. the subject gives an indication of, or feels, an effect).

More preferred compositions of the invention may be adapted (for example as described herein) to provide a sufficient dose of agent over the dosing interval (irrespective of the number of doses per unit time) to produce a desired therapeutic effect.

The amounts of agent that may be employed in compositions of the invention may thus be determined by therapeutic standards, the physician, or the skilled person, in relation to what will be most suitable for an individual patient or the condition to be treated. This is likely to vary with the type and severity of the condition that is to be treated, as well as the age, weight, sex, renal function, hepatic function and response of the particular patient to be treated.

Suitable daily dosages of nicotine, both for medicinal and recreational purposes, may be from about 1 to about 100 mg/day. For example, the total amount of nicotine or salt thereof contained within the composition may be from about 0.5 mg to about 15 mg nicotine calculated as the free base form. Conventional cigarettes typically contain between about 8 and 15 mg nicotine. In one embodiment, each nicotine pouch or tablet of the invention is capable of delivering from about 3 to about 15 mg nicotine. It is preferred that the products disclosed herein are capable of delivering an amount of nicotine that is at least equivalent to one cigarette. When the nicotine is supplied to the patient in the form of a tablet (e.g. a sublingual or buccal tablet), then each tablet may contain from about 0.5 to about 15 mg nicotine, e.g. from about 8 mg to about 15 mg of nicotine.

Suitable dosages of other active pharmaceutical agents in one oral delivery unit (e.g. one tablet) may be below 1 g, preferably below 100 mg and above 1 μg.

When compositions of the invention comprise opioid analgesics, appropriate pharmacologically effective amounts of such opioid analgesic compounds include those that are capable of producing (e.g. sustained) relief of pain when administered intraorally. Thus, the total amount of opioid analgesic active ingredient that may be employed in a composition of the invention will depend upon the nature of the relevant active ingredient that is employed, but may be in the range of about 0.0005%, such as about 0.1% (e.g. about 1%, such as about 2%) to about 20%, such as about 10%, for example about 7%, by weight based upon the total weight of the composition. The amount of this active ingredient may also be expressed as the amount in a unit dosage form. In such a case, the amount of opioid analgesic active ingredient that may be present may be sufficient to provide a dose per unit dosage form that is in the range of between about 1 μg (e.g. about 5 μg) and about 50 mg (e.g. about 15 mg, such as about 10 mg).

The above-mentioned dosages are exemplary of the average case; there can, of course, be individual instances where higher or lower dosage ranges are merited, and such are within the scope of this invention.

Compositions of the invention comprising opioid analgesics are useful in the treatment of pain, particularly severe and/or chronic pain. According to a further aspect of the invention there is provided a method of treatment of pain which method comprises administration of a composition of the invention to a person suffering from, or susceptible to, such a condition.

For the avoidance of doubt, by “treatment” we include the therapeutic treatment, as well as the symptomatic treatment, the prophylaxis, or the diagnosis, of the condition.

When used for recreational purposes, the formulations are capable of delivering a sufficient amount of nicotine to provide a pleasurable sensory experience for the user.

The compositions of the invention are formed from calcium salts and so may also be beneficial for a user's oral health. Chemically bonded ceramic systems that are capable of releasing calcium (e.g. in the form of solubilised calcium ions) into saliva are capable of remineralization and so can, for example, contribute to mineral growth on the surfaces of teeth. Materials that allow excess ions (e.g. calcium ions, hydroxide ions and possibly also phosphate ions) to diffuse into surrounding saliva under physiological conditions (e.g. at the pH and temperature typically found in the mouth) may be particularly suited for this, and such materials include chemically bonded ceramic systems formed from alpha-tricalcium phosphate and tetracalcium phosphate. Chemically bonded ceramic system that are formed from materials other than calcium phosphates but which are still capable of releasing calcium into saliva are also useful in this context as saliva can act as a source of phosphate ions, thus contributing to the growth of minerals, such as apatite, on teeth. Such materials include chemically bonded ceramic systems formed from calcium silicates, calcium aluminates and, particularly, calcium sulphate. Intraoral compositions of the invention therefore have potential utility in repairing dental enamel and strengthening teeth.

The formulations of the invention may have the advantage that they provide an extended release of nicotine (e.g. nicotine is released at a sufficiently retarded rate to produce a therapeutic response or give a pleasurable experience over an extended period of time compared to pouch formulations currently on the market). The formulations of the invention may also have the advantage that they may be more efficacious than, be less toxic than, be faster acting than, be more potent than, produce fewer side effects than, be more easily absorbed than, and/or have a better pharmacokinetic profile than, have improved bioavailability over, and/or have other useful pharmacological, physical, or chemical properties over, pharmaceutical compositions known in the prior art.

The use of the chemically bonded ceramic systems described herein affords for the provision of products which provide acceptable levels of release of nicotine when placed in the mouth, while minimising the risk of exposure to the stored nicotine (or salt thereof) within, e.g. through leakage.

The chemically bonded ceramic systems are also easily manufactured without the need for high temperature sintering, and therefore additional elements such as flavours, fillers and the like can be incorporated into the carrier to aid in the achieving the desired sensory experience. The ability to incorporate the nicotine (or salt thereof) into the carrier as the network structure is formed also allows for greater control over the amount of nicotine that is ultimately delivered to the user.

Oral products, particularly nicotine pouches, containing the compositions of the invention may also be suitable for storage under ambient temperature without significant degradation of the components, e.g. the alkaline bioceramic, contained within. Avoiding the need for refrigerated storage is of clear benefit for manufacturers, retailers and users of these products.

Wherever the word “about” is employed herein in the context of dimensions (e.g. values, temperatures, pressures (exerted forces), relative humidities, sizes and weights, particle or grain sizes, pore sizes, timeframes etc.), amounts (e.g. relative amounts (e.g. numbers or percentages) of particles, individual constituents in a composition or a component of a composition and absolute amounts, such as doses of nicotine, numbers of particles, etc.), deviations (from constants, degrees of degradation, etc.) it will be appreciated that such variables are approximate and as such may vary by ±10%, for example ±5% and preferably t 2% (e.g. ±1%) from the numbers specified herein.

### THE INVENTION IS ILLUSTRATED BY THE FOLLOWING EXAMPLES IN WHICH

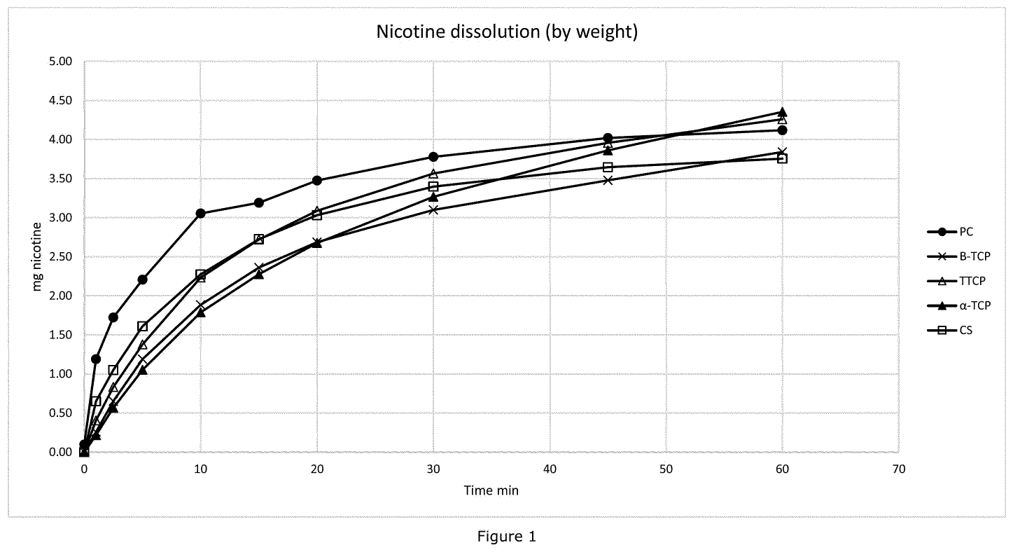

FIG. 1 shows the nicotine release profiles (by weight) for different nicotine-loaded alkaline bioceramics;

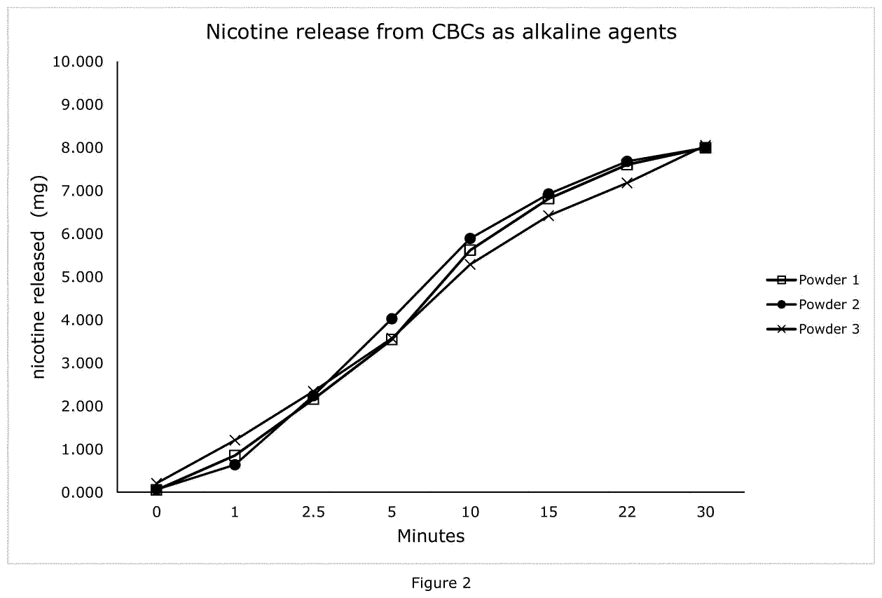

FIG. 2 shows the nicotine release profiles for a mixture of an alkaline bioceramic pH regulator and calcium sulphate loaded with nicotine;

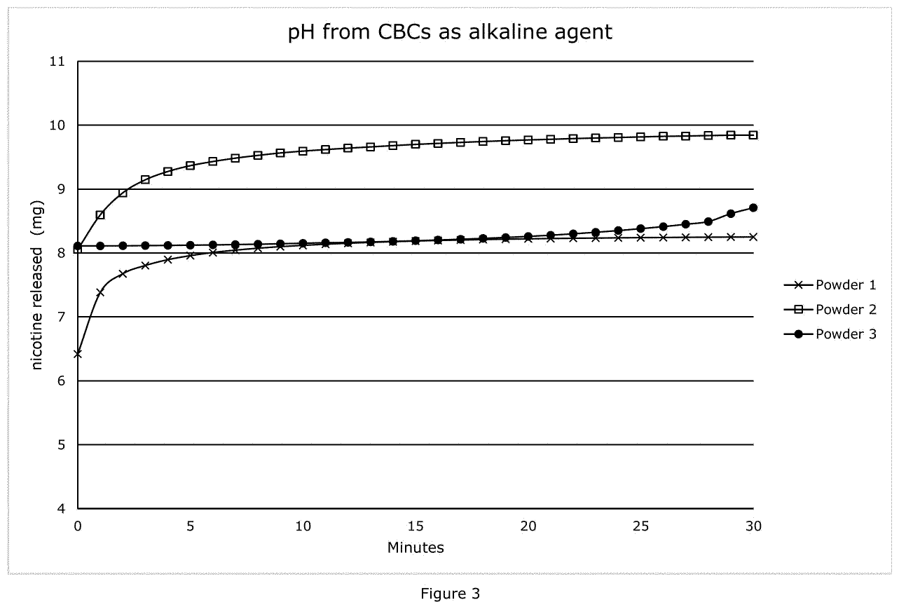

FIG. 3 shows the pH profiles for a mixture of an alkaline bioceramic pH regulator and calcium sulphate loaded with nicotine;

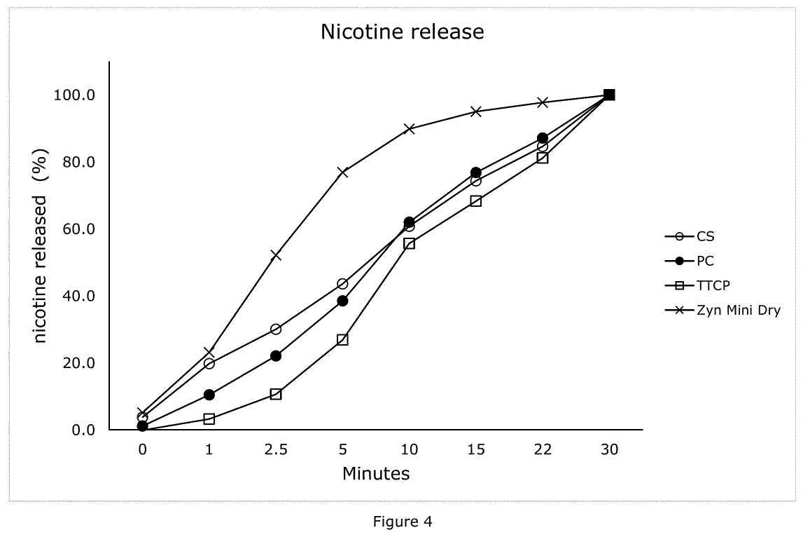

FIG. 4 shows the release profiles for alkaline bioceramics loaded with nicotine in comparison to a commercial product;

FIG. 56 shows the pH profiles for alkaline bioceramics loaded with nicotine in comparison to a commercial product;

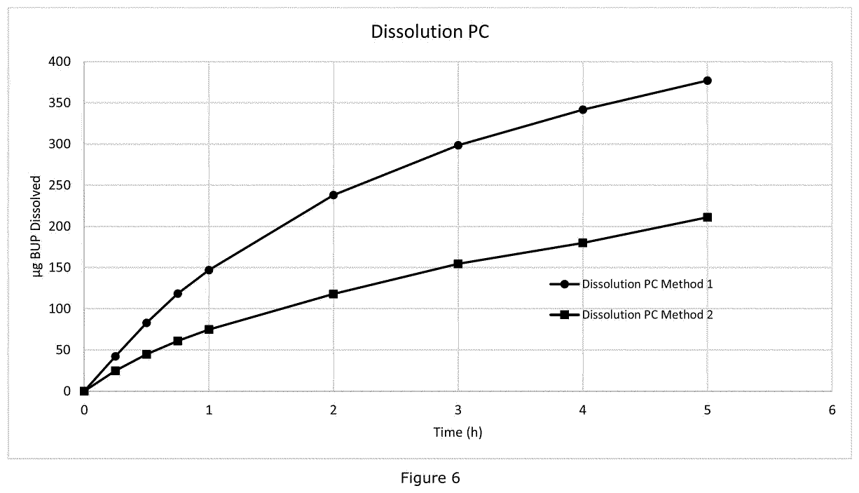

FIG. 6 shows the buprenorphine release profile for Portland cement loaded with BUP HCl;

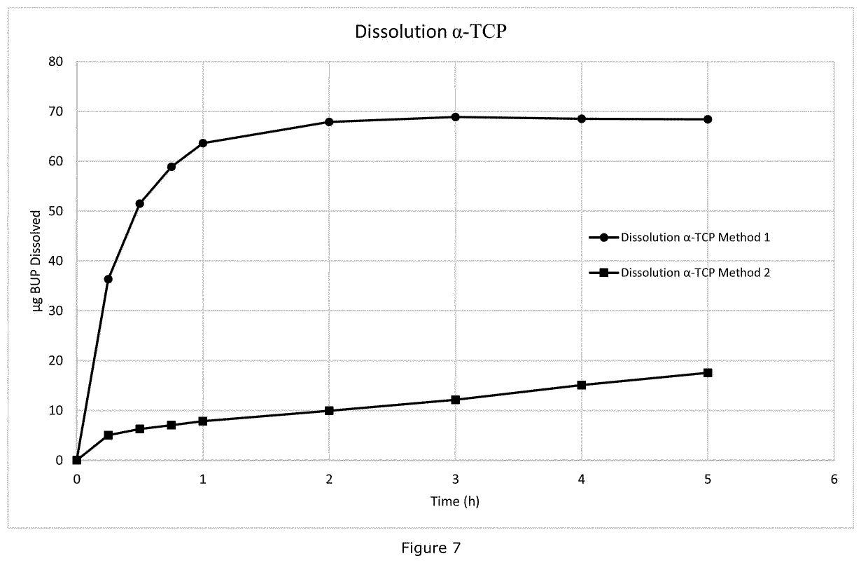

FIG. 7 shows the buprenorphine release profile for α-TCP loaded with BUP HCl.

### EXAMPLES

### Example 1—Bioceramics as Carrier and pH Regulator

Bioceramic powders containing nicotine were prepared using different chemically bonded ceramics and the nicotine dissolution and pH was measured.

### Materials

USP grade nicotine bitartrate dihydrate
De-ionised water
(CS) Calcium Silicate powder (CaO/SiO₂=1/1)
(PC) Portland cement
(TTCP) Tetra calcium phosphate
(α-TCP) Alpha Tricalcium Phosphate
(β-TCP) Beta Tricalcium phosphate

### Method

First an evaluation was done to find the appropriate water to cement (w/c) ratio to give a good setting material. The evaluation was done by manually mixing the raw material with de-ionized water at different w/c ratios and analyzing the setting time. The resulting w/c ratios for respective bioceramic used for further evaluation can be found in Table 1.

TABLE 1
The appropriate w/c ratio to give good setting
characteristics for respective bioceramic

| | Appropriate w/c |
|---|---|
| Bioceramic | ratio |
| CS | 0.5 |
| PC | 0.4 |
| TTCP | 0.3 |
| α-TCP | 0.3 |
| β-TCP | 0.8 |

Nicotine bitartrate dihydrate salt was dissolved in de-ionised water. One solution for each bioceramic with a concentration calculated to give a set material with a nicotine content of 6 mg per 0.3 g of powder. The bioceramic materials was mixed manually using a spatula in a glass beaker with the respective nicotine solutions according to the w/c ratios in Table 1. The resulting materials were left to set and dry on an aluminum foil at ambient room conditions for at least 1 hour. Thereafter the materials were crushed using a pestle and mortar. The crushed powders were filled into pouches of a non-woven heat sealable material and closed by heat welding. 0.3 g of powder was added to each pouch.

### Analysis

To measure nicotine release and pH, each pouch was suspended in a met al net cage in 100 ml of distilled water in a glass beaker containing a stirrer magnet. The beaker was placed on a magnetic stirrer at a stirrer speed of 360 rpm. For nicotine content samples were taken out at different time points using a pipette and put into sealable vials for analysis. The pH was measured continuously throughout the experiment by having a pH probe submerged in the sample beaker. The pH meter used was a calibrated Mettler Toledo Seven compact pH/Ion S220 system with a Mettler Toledo InLab Expert Pro electrode. For nicotine content the absorbance of the analytes was subsequently measured using a UV-spectrophotometer system (VWR UV-3100PC) at 260 nm wavelength. The absorbance of each analyte was calculated as nicotine concentration using a calibration curve and the known powder weight in each pouch.

### Results

The resulting pH after 30 min of stirring on the magnetic stirrer can be seen in Table 2.

TABLE 2

| Bioceramic (with | |
|---|---|
| nicotine) | pH |
| CS | 9.5 |
| PC | 11.0 |
| TTCP | 8.4 |
| α-TCP | 7.8 |
| β-TCP | 6.3 |

The mean (n=3) dissolution of nicotine from respective bioceramic presented as mg nicotine and as % of the 60 min value is shown in FIG. 1. All of the bioceramics demonstrated good release profiles.

### Example 2—Different Carrier and pH Regulator

This example illustrates using Calcium Silicate, Portland cement and sodium carbonate as pH regulator added to a Calcium Sulphate based nicotine powder.

### Materials

USP grade nicotine bitartrate dihydrate
De-ionised water
(CSH) BP, DAB grade Calcium sulphate Hemihydrate

$$( CS ) Calcium Silicate powder ( CaO / SiO 2 = 1 / 1 )$$

(PC) Portland cement
(SC) Sodium Carbonate

### Method

A nicotine-loaded base powder was made using BP and DAB grade calcium sulphate hemihydrate (CaSH), and nicotine in the form of USP grade nicotine bitartrate dihydrate in according to the following method:

170.4 g of Nicotine Bitartrate salt was dissolved in 1215.7 g of de-ionized water in a household blender. 1519.6 g of CaSH was added in portions. The mix was blended for about 3 min using medium speed on the blender. Thereafter the blend was poured onto a silicon canvas with met al walls and left to set, creating a roughly 10 mm thick plate. The plate was then put onto a met al plate with holes and left to dry at room temperature for roughly 24 h. The casted plate was then broken up into smaller pieces using pestle and mortar and finally ground in a household flour mill. The resulting powder was then sieved using a Retsch® AS 200 Basic sieve shaker stack to obtain particles in the range of 50-500 μm. The resulting powder (“Nicotine-loaded base powder”) was then mixed with the CS and PC, respectively, to generate two different powders. In addition one powder was made where the alkaline agent used was the standard agent Sodium Carbonate, powder 3.
Powder 1:1 g of Nicotine-loaded base powder was mixed with 5 wt % of CS
Powder 2:1 g of Nicotine-loaded base powder was mixed with 3.5 wt % of PC
Powder 3:1 g of Nicotine-loaded base powder was mixed with 3.5 wt % of SC

The powders were mixed by hand using a spatula in a beaker to create a homogenous blend. 0.3g of the resulting powders was respectively put into a standard size pouch made of non-woven material for analysis.

### Analysis

The pouches were suspended in a steelwire “cage” in a beaker with 50 mL of deionised water. A magnet was added to the beaker that was immediately placed on a magnetic stirrer.

pH

The pH measurements were performed using a calibrated Mettler Toledo Seven compact pH/Ion S220 system with a Mettler Toledo InLab Expert Pro electrode. The pH-meter was set to automated measurements and the pH was measured once every minute.

### Nicotine Release

Samples was withdrawn manually using a pipette and the samples were then analysed with a UV-spectrophotometer (VWR UV-3100PC) at a wavelength of 260 nm. Standard curves were obtained using defined solutions with nicotine base.

### Results

The resulting nicotine release and pH profiles can be seen in FIGS. 2 and 3, respectively.

### Example 3—Nicotine Release and pH Profile Comparison

The pH development and nicotine release of the three powders according to the invention was compared to the pH development of the commercial product Zyn® Mini Dry.

### Materials

USP grade nicotine bitartrate dihydrate
De-ionised water

$$( CS ) Calcium Silicate powder ( CaO / SiO 2 = 1 / 1 )$$

(PC) Portland cement
(TTCP) Tetra calcium phosphate
Zyn® Mini Dry (as commercially available)

### Method

The Nicotine bitartrate dihydrate salt was dissolved in the de-ionised water to give a solution with a nicotine concentration of 60 mg/ml.

Powder 1:1g of CS was weighed into a beaker. 0.4 ml of the nicotine solution was added and the mixture was stirred with a spatula for 1 min. The mixture was left to set for 5 min and then transferred onto aluminium foil and left to “dry” in room temperature for 1 hour. Thereafter the hardened cement was crushed using a pestle and mortar. 0.3g of the resulting powder was put into a standard size pouch made of non-woven material for analysis.

Powder 2: For the Portland cement product, the exact same method as for CS was used to produce samples, using PC in place of CS.

Powder 3: A TTCP product was prepared using the same method as the CS product except that the w/c (water:cement) ratio was changed to 0.6 instead of 0.4. Accordingly, the nicotine solution was diluted with more de-ionised water to give the same amount of nicotine in the mixed cement.

Pouches with 0.3g of powders 1, 2 and 3 were prepared. The commercial products were used directly from their commercial packaging.

### Analysis

The same method for pH analysis and nicotine release was used as in Example 2.

### Results

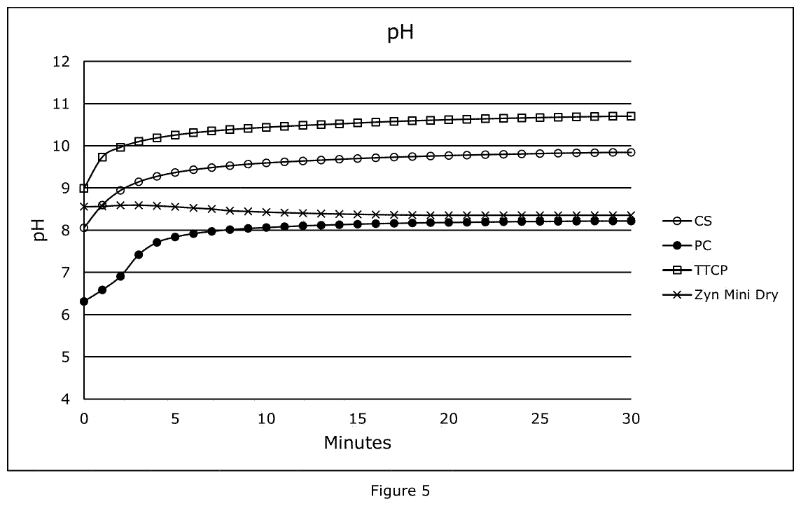

The result can be seen in FIGS. 4 and 5.

### Example 4—Moist Formulation

A moist formulation similar in texture, consistency and moistness to a commercially available moist (Zyn® Slim) product is made.

The powder from Example 3 is mixed with 10, 15 and 20 wt % of Propylene Glycol (PG) in a beaker using a spatula. The resulting mixtures all have texture, consistency and moistness comparable to commercially available moist products although there is a difference between them in terms of how moist and coherent they are. Pouches with 0.3g of each mixture are prepared using non-woven fabric.

### Analysis

The same method for pH analysis and nicotine release is used as in Example 1.

Results pH and nicotine release is expected to be similar to the dry powders tested in Example 1.

### Example 5—Evaluation of User Experience

A small scale subjective evaluation with Nicotine pouch users (n=10) is performed. Each user is given a can with pouches produced according to Example 3 with two different flavours one Citrus and one Mint. Only the CS and PC powders of example 3 are tested giving four test powders (Powders 1-4). The flavours are added to the powders by hand mixing of 3 wt % of a flavour liquid into the remaining components of the powders. Each subject is also given one can of Dry (Zyn® Mini Dry) and one can of Moist (Zyn® Slim) commercial competitive product with Mint flavour for comparison. The subjects is asked to rate the smell in the can, the speed of nicotine onset, the duration of nicotine release, the speed of flavour onset and the duration of flavour release.

Powders 1-4 are expected to have improved or comparable performance compared to the commercial products.

### Example 6—Storage Stability

The stability of test materials under humid conditions is assayed using the following method.

### Test Material

A powder prepared according to Example 3 is used as the test formulations. Commercial dry and moist formulations are used for comparison. The formulations are provided in the form of powders for storage which are then sealed in pouches made of non-woven cellulose prior to storage.

### Storage Conditions

The sealed pouches are stored in under the following conditions:

Test duration: at least 4 weeks days, optionally longer.

Vessel/container: in open pouch cans without lid. Cans placed in larger sealed plastic containers with different humidity.

Temperature: room temperature. Typically varying between 20° C. and 23° C.

Humidity: Two different levels of relative humidity (“RH”) are used: 33% and 60% RH. The RH is controlled by having saturated salt solutions with different salts in the bottom of the plastic containers, as described in Greenspan L., Journal of Research of the National Bureau of Standards, vol. 81A, No. 1, 1977, 89-96.

### Analysis

Analysis of the nicotine content before and after storage is made using the UV-spectrophotometry method in Example 1. pH measurements are made using the method in Example 2.

### Example 7—Buprenorphine

Other substances besides nicotine can be incorporated into and subsequently released from the CBCs (chemically bonded ceramic systems).

### Materials

USP grade Buprenorphine Hydrochloride
De-ionised water

$$( CS ) Calcium Silicate powder ( CaO / SiO 2 = 1 / 1 )$$

(PC) Portland cement
(TTCP) Tetra calcium phosphate

### Method

Buprenorphine hydrochloride (BUP-HCL) is dissolved in the de-ionised water to give a solution with a buprenorphine concentration of 19 mg BUP/ml. Note that the conversion of BUP-HCL to BUP is 0,928 so the theoretical BUP-HCL concentration would be about 20.5 mg/ml.

1g of CS is weighed into a beaker. 0.4 ml of the BUP solution is added and the mixture is stirred with a spatula for 1 min. The mixture is left to set for 5 min and then transferred onto an aluminium foil and left to “dry” in room temperature for 1 hour. Thereafter the hardened cement is crushed using a pestle and mortar. 0.3g of the resulting powder is put into a standard size pouch made of non-woven material for analysis.

For the Portland cement product, the exact same method as for CS is used to produce samples, using PC in place of CS.

A TTCP product is prepared using the same method as the CS product except that the w/c (water:cement) ratio was changed to 0.6 instead of 0.4. Accordingly the BUP solution is diluted with more de-ionised water to give the same amount of BUP in the mixed cement.

### Analysis

The BUP containing powders are tested by dissolution testing according to the following. For the dissolution testing a USP apparatus 2, Pharma Test PTWS 120D is used. 100 ml (±1%) pre-heated (37±0,5° C.) dissolution medium (50 mM phosphate buffer pH 6.8) is added into each dissolution vessel (n=6). One pouch is added to each vessel at time=0 and stirring starts (50 rpm). The pouches are allowed to drop to the bottom of the vessel before the paddle rotation is activated. 1 ml are withdrawn using a syringe at t=0.25, 0.50, 0.75, 1, 2, 3, 4, 5, 6, 7 and 24 hours. The withdrawn samples are transferred to microcentrifuge tubes (1.5 ml).

The samples is acidified with 0.2 M HCl; 800 μl sample+200 μl 0.2 M HCl is pipetted directly into a HPLC vial and mixed.

The samples are analysed with a HPLC system set up according to:
HPLC instrument Shimadzu Prominence-i with UV-detector, LC-2030
Chromatography software LabSolutions v. 5.99
Analytical column Sunniest C18, 3 μm, 2.0×100 mm (ChromaNik Technologies)
Pre-column SecurityGuard Cartridge Gemini-NX C18 2.0 mm id (Phenomenex)
Pump A 10 mM phosphate buffer pH 2.5
Pump B Acetonitrile
Mobile phase 71% 10 mM phosphate buffer pH 2.5 (A): 29% ACN (B)
Flow rate 0.2 ml/min
Column temperature+30° C.
Autosampler temperature Ambient
Injection volume 5 μl
UV detection 211 nm
Run time 9 min

### Results

Buprenorphine release in detectable amounts for a duration of at least 30 minutes is expected from all three powders.

### Example 8—Portland Cement Product

Hardened Portland cement was used as pH regulator with a calcium phosphate nicotine granulate.

### Method

### Sample Preparation

### Step 1:

### Manufacturing of Calcium Phosphate Nicotine Granules

153 g USP grade Nicotine bitartrate dihydrate was dissolved in 430 ml of distilled water. 1973 g of DAB/BP grade Calcium sulphate hemihydrate was weighed into the steel drum of an intensive mixer type granulator. The granulator was started at a speed of 20 m/s and the nicotine/water solution was poured in during 15 s. The granulator was on for another 15 s whereafter the speed was lowered to half and run for another 120 s before turned off and the material was checked. The granulator was turned on again at 20 m/s speed, another 100 ml of water was added, and the machine run for 15 s and turned off. The granulator was started again, another 150 ml of water added, and the granulator was on for 15 s. The resulting granulate was poured onto an Al-tray and dried at room temperature for 24 hours. The dried granulate was sieved using a Retsch® AS 200 Basic sieve shaker stack to obtain particles in the range of 50-500 μm. The resulting nicotine content in the powder was approximately 6 mg of nicotine/0.3 g of powder.

### Step 2:

### Manufacturing of the Hardened Portland Cement Powder

10 Portland cement and 4g water was mixed manually in a glass beaker into a homogeneous paste. The paste was then left to set and harden on an Al-foil at ambient room conditions for at least 1 hour. Thereafter the material was crushed manually using a pestle and mortar. The crushed powder was sieved using a Retsch® AS 200 Basic sieve shaker stack to obtain particles in the range of 50-500 μm.

### Step 3:

Mixing of Hardened Portland cement with Nicotine containing Calcium Phosphate granules.

20 g of the granules from step 1 was mixed with four different amounts of hardened Portland cement powder according to Table 3. Mixing was done in a Turbula® dry mixer for 30 min at 50 rpm.

TABLE 3
Four powders was prepared with varying wt % of hardened PC

| | Wt % hardened |
|---|---|
| Sample | PC |
| Powder 1 | 1 |
| Powder 2 | 2 |
| Powder 3 | 3 |
| Powder 4 | 5 |

### Step 4

Filling Powder into Pouches

0.3 g of powder was filled into pouches of a non-woven heat sealable material designed for use as nicotine pouches. Three pouches for powders 2, 3 and 4 in Table 3 were made.

### Analysis

Three different sets of measurements were performed with the different samples, two different pH measurements and one dissolution experiment.

pH method 1:

0.3g of powders 1, 2 and 4, according to Table 3 was added to 100 ml of de-ionised water in a glass beaker with a stirring magnet. The beaker was placed on a magnetic stirrer at 360 rpm. After 30 minutes pH was measured using a calibrated Mettler Toledo Seven compact pH/Ion S220 system with a Mettler Toledo InLab Expert Pro electrode. Three samples of each powder were analysed.

pH method 2:

The filled pouches, with powders 2, 3 and 4 was suspended in a met al net cage in 100 ml of de-ionised water in a glass beaker containing a stirrer magnet. The beaker was placed on a magnetic stirrer at a stirrer speed of 360 rpm. After 30 minutes pH was measured using a calibrated Mettler Toledo Seven compact pH/Ion S220 system with a Mettler Toledo InLab Expert Pro electrode. Three samples of each powder were analysed.

### Dissolution:

Pouches filled with 0.3 g of powder 3 according to Table 3, were suspended in a met al net cage in 100 ml of de-ionised water in a glass beaker containing a stirrer magnet. The beaker was placed on a magnetic stirrer at a stirrer speed of 360 rpm. For dissolution analysis samples were taken after 30 minutes using a pipette and put into sealable vials for analysis. The samples were then analyzed by measuring absorbance using a UV-spectrophotometer system (VWR UV-3100PC) at 260 nm wavelength. The absorbance of each analyte was calculated as nicotine concentration using a calibration curve and the known powder weight. Three pouches were analyzed.

### Results

The results of the pH measurements are presented in Table 4 as a mean of three samples. A desirable pH of 8 or above could be achieved with powders 3 and 4.

TABLE 4
Results from the two different pH methods

| Material | pH (pH method 1) | pH (pH method 2) |
|---|---|---|
| Powder 1 | 5.4 | |
| Powder 2 | 7.6 | 7.6 |
| Powder 3 | | 8.1 |
| Powder 4 | 10.1 | 8.6 |

The results from the dissolution experiment can be seen in Table 5. It is confirmed that at least 6 mg of nicotine was dissolved from each sample confirming that the addition of hardened PC powder does not disturb the nicotine release.

TABLE 5
Amount of dissolved nicotine in the dissolution experiment.

| | Dissolved nicotine |
|---|---|
| Pouch | (mg) |
| 1 | 6.3 |
| 2 | 6.1 |
| 3 | 6.1 |

### Example 9—Example Bioceramic Carrier with Buprenorphine Hydrochloride

Two bioceramic materials loaded with buprenorphine hydrochloride were made and dissolution and pH was tested.

### Method

### Sample Manufacturing

### Step 1

### Powder Mixing

4.996g of white Portland cement (PC) and 0.03507g of USP grade Buprenorphine hydrochloride (BUP-HCl) was weighed into a 30 ml amber glass jar by manual volume dilution using a spatula. The mouth of the jar was covered with a thin PE-foil and the lid was put on and closed tight. The closed jar was put into a Turbula dry mixer and mixed at 47 rpm for 40 minutes.

4.994g of α-TCP (Tri Calcium Phosphate) cement and 0.03275g of USP grade Buprenorphine hydrochloride (BUP-HCl) was weighed into a 30 ml amber glass jar by manual volume dilution using a spatula. The mouth of the jar was covered with a thin PE-foil and the lid was put on and closed tight. The closed jar was put into a Turbula dry mixer and mixed at 47 rpm for 40 minutes.

### Step 2

### Powder Water Mixing and Hardening

The PC-BUP-HCL powder was mixed with de-ionised water at a water to Portland cement (w/c) ratio of 0.4 by adding 2 ml of water into the jar with powder. The mixture was stirred manually using a spatula for two minutes and the resulting paste was put into a standard snuff can covered with Al-foil. The lid was put on and the paste was left to set and harden over night, approximately 17 hours.

The α-TCP-BUP-HCL powder was mixed with de-ionised water at a water to Tri Calcium Phosphate (w/c) ratio of 0.3 by adding 1.5 ml of water into the jar with powder. The mixture was stirred manually using a spatula for two minutes and the resulting paste was put into a standard snuff can covered with Al-foil. The lid was put on and the paste was left to set and harden over night, approximately 17 hours

### Step 3:

### Crushing and Pouch Filling

The set and hardened materials were crushed manually using a pestle and mortar. 0.3g of powder was weighed into each pouch of non-woven heat sealable material designed for use as nicotine pouches and sealed using a manual heat sealer. Six pouches of each material were produced.

### Analysis

Two different dissolution methods were employed. Three pouches of each material were tested in respective method.

Dissolution method 1:

Each pouch was suspended in a met al net cage in 100 ml of de-ionised water in a glass beaker containing a stirrer magnet. The beaker was placed on a magnetic stirrer at a stirrer speed of 360 rpm. Samples were taken out at different time points using a pipette and put into sealable vials for analysis.

### Dissolution Method 2:

A USP 2 apparatus with mini vessels operating at 50 rpm and 37° C. was used for this method. Each vessel contained 100 ml of de-ionised water. The pouches were put into the vessels, one pouch in each vessel. In order to keep the pouches below the water surface each pouch was first put into a steel wire cage. Samples were taken out at different time points using the built in sample withdrawal unit on the apparatus and put into sealable vials for analysis.

### HPLC Analysis

In order to evaluate the amount of BUP dissolved from the pouches the samples withdrawn from the dissolution experiments were analysed in a Shimadzu Prominence-I LC-2030 HPLC system with a UV detector at 211 nm.

### pH Analysis

The resulting pH in the dissolution baths were measured using a calibrated Mettler Toledo Seven compact pH/Ion S220 system with a Mettler Toledo InLab Expert Pro electrode.

### Results

The results from the pH measurements can be seen in Table 6 and the results from the dissolution experiments can be seen in FIGS. 6 and 7.

TABLE 6

| | pH after dissolution | pH after dissolution |
|---|---|---|
| Bioceramic | with method 1 | with method 2 |
| PC | 11.8 | 11.4 |
| α-TCP | 7.8 | 7.5 |

A clear difference in dissolution rate can be seen between Bioceramics and dissolution methods. However, in all cases BUP was released from the Bioceramics.

1. An intraoral composition comprising an alkaline active pharmaceutical agent, or a salt thereof, and a chemically bonded ceramic system formed from a substance selected from the group consisting of calcium aluminates, calcium silicates, calcium phosphates and mixtures thereof.

2. The composition according to claim 1, wherein the composition gives a pH of at least 8 upon contact with saliva.

3. The composition according to claim 1 or claim 2, wherein the alkaline active pharmaceutical agent is an active pharmaceutical agent with a pKₐ in the range of 7 to 10.

4. The composition according to claim 3, wherein the agent is nicotine or a salt thereof.

5. The composition according to claim 4, wherein the composition is prepared using a salt of nicotine, optionally wherein the salt is a nicotine bitartrate salt, such as nicotine bitartrate dihydrate.

6. The composition according to claim 4 or claim 5, wherein the total amount of nicotine or salt thereof contained within the composition is from about 0.5 mg to about 15 mg nicotine calculated as the free base form.

7. The composition according to claim 1 or claim 2, wherein the alkaline active pharmaceutical agent is an opioid analgesic.

8. The composition according to claim 1 or claim 2, wherein the active agent is selected from the group consisting of morphine, codeine, thebaine or a Diels-Alder adduct thereof, diamorphine, hydromorphone, oxymorphone, hydrocodone, oxycodone, etorphine, nicomorphine, hydrocodeine, dihydrocodeine, metopon, normorphine, N-(2-phenylethyl)normorphine, racemorphan, levorphanol, dextromethorphan, levallorphan, cyclorphan, butorphanol, nalbufine, cyclazocine, pentazocine, phenazocine, pethidine (meperidine), fentanyl, alfentanil, sufentanil, remifentanil, ketobemidone, carfentanyl, anileridine, piminodine, ethoheptazine, alphaprodine, betaprodine, 1-methyl-4-phenyl-1,2,3,6-tetrahydropyridine, diphenoxylate, loperamide, methadone, isomethadone, propoxyphene, levomethadyl acetate hydrochloride, dextromoramide, piritramide, bezitramide, dextropropoxyphene, buprenorphine, nalorphine, oxilorphan, tilidine, tramadol, dezocine, allylprodine, benzylmorphine, clonitazene, desomorphine, diampromide, dihydromorphine, dimenoxadol, dimepheptanol, dimethylthiambutene, dioxaphetyl butyrate, dipipanone, eptazocine, ethylmethylthiambutene, ethylmorphine, etonitazene, hydroxypethidine, levophenacylmorphan, lofentanil, meptazinol, metazocine, myrophine, narceine, norpipanone, papaveretum, phenadoxone, phenomorphan, phenoperidine and propiram.

9. The composition according to any one of the preceding claims, wherein the chemically bonded ceramic system is formed by hydration of a substance selected from the group consisting of CaOAl₂O₃, (CaO)₁₂(Al₂O₃)₇, (CaO)₃(Al₂O₃), (CaO)(Al₂O₃)₂, alpha-tricalcium phosphate, tetracalcium phosphate, (CaO)₃(SiO₂), and (CaO)₂(SiO₂).

10. The composition according to any one of the preceding claims, wherein the chemically bonded ceramic system is present at from about 0.1% to about 50% by weight of the composition.

11. The composition according to any one of the preceding claims, wherein the porosity of the chemically bonded ceramic system is from about 10% to about 70%.

12. The composition according to any one of the preceding claims, wherein a portion of the alkaline active pharmaceutical agent or salt thereof, such as at least 20% by weight, is located within pores in the chemically bonded ceramic system.

13. The composition according to any one of the preceding claims, wherein the composition is capable of releasing substantially all of the alkaline active pharmaceutical agent or salt thereof upon contact with an aqueous liquid.

14. The composition according to any one of the preceding claims, wherein sodium carbonate and sodium bicarbonate are substantially absent from the composition.

15. The composition according to any one of the preceding claims, wherein the composition is provided in the form of a permeable, sealed bag containing the solid, porous chemically bonded ceramic system and the alkaline active pharmaceutical agent or salt thereof.

16. The composition according to claim 15, wherein the bag further contains a filler, flavour or controlled release agent.

17. The composition according to any one of claims 1 to 13, wherein the composition is a sublingual tablet, buccal tablet, wafer or lozenge.

18. The composition according to claim 17 further comprising a bioadhesion and/or mucoadhesion promoting agent.

19. A method of forming an intraoral composition as defined in any one of the preceding claims, wherein the method comprises bringing an alkaline active pharmaceutical agent or salt thereof into association with a chemically bonded ceramic system formed from a substance selected from the group consisting of calcium aluminates, calcium silicates, calcium phosphates, and mixtures thereof.

20. A method of treating nicotine dependence, treating one or more symptoms of nicotine dependence, aiding smoking cessation or ameliorating symptoms associated with a disease or conditions selected from the group consisting of dementia, Alzheimers disease, Parkinson s disease, Huntington s disease and depression, which method comprises administering an intraoral composition as defined in any one of claims 3 to 6 or 9 to 18 to a person in need thereof.

21. A method of treatment of pain which comprises administration of a composition as defined in any one of claims 7 to 18, to a person suffering from, or susceptible to, such a condition.
